SEQUENCE LISTING (47 sequences)

SEQ ID NO: 1 - protein, 209 aa, Escherichia coli MG1655
MVDMKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 2 - DNA, 630 bp, Escherichia coli MG1655
atggtcgatatgaaaactacgcatacctccctcccctttgccggacatacgctgcattttgttgagttcgatccggcgaatttttgtgagcaggatttactctggctgccgcactacgcacaactgcaacacgctggacgtaaacgtaaaacagagcatttagccggacggatcgctgctgtttatgctttgcgggaatatggctataaatgtgtgcccgcaatcggcgagctacgccaacctgtctggcctgcggaggtatacggcagtattagccactgtgggactacggcattagccgtggtatctcgtcaaccgattggcattgatatagaagaaattttttctgtacaaaccgcaagagaattgacagacaacattattacaccagcggaacacgagcgactcgcagactgcggtttagccttttctctggcgctgacactggcattttccgccaaagagagcgcatttaaggcaagtgagatccaaactgatgcaggttttctggactatcagataattagctggaataaacagcaggtcatcattcatcgtgagaatgagatgtttgctgtgcactggcagataaaagaaaagatagtcataacgctgtgccaacacgattaa

SEQ ID NO: 3 - protein, 209 aa, Escherichia coli O157H7 EDL933
MVDMKTTHTSLPFAGHTLHFVEFDPANFCEQDLLWLPHYAQLQHAGRKRKTEHLAGRIAAVYALREYGYKCVPAIGELRQPVWPAEVYGSISHCGATALAVVSRQPIGVDIEEIFSAQTATELTDNIITPAEHERLADCGLAFSLALTLAFSAKESAFKASEIQTDAGFLDYQIISWNKQQVIIHRENEMFAVHWQIKEKIVITLCQHD

SEQ ID NO: 4 - protein, 209 aa, Shigella sonnei Ss046
MVDMKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASERQTEAGFLDYQIISWNKQQVIIHRENEMFAVHWQIKEKIVITLCQHD

SEQ ID NO: 5 - protein, 256 aa, Shigella flexneri 5 str. 8401
MRVVHAGCGVNALSGLQRSCQFNILQDHVGLISVAHQAVLRLSSVSNMVDMKTTHTSLPFAGHTLHFVEFDPANFCEQDLLWLPHYAQLQHAGRKRKTEHLAGRIAAVYALREYGYKCVPAIGELRQPVWPAEVYGSISHCGATALAVVSRQPIGVDIEEIFSAQTATELTDNIITPAEHERLADCGLAFSLALTLAFSAKESAFKASEIQTDAGFLDYQIISWNKQQVIIHRENEMFAVHWQIKEKIVITLCQHD

SEQ ID NO: 6 - protein, 209 aa, Shigella boydii Sb227
MVDMKTTHTSLPFAGHTLHFVEFDPANFCEQDLLWLPHYAQLQHAGRKRKAEHLAGRIAAIYALREYGYKCVPAIGELRQPVWPAEVYGSISHCGATALAVVSRQPIGVDIEEIFSAQTATELTDNIITPAEHERLADCGLAFSLALTLAFSAKESAFKASEIQTDAGFLDYQIISWNKQQVIIHRENEMFAVHWQIKEKIVITLCQHD

SEQ ID NO: 7 - protein, 209 aa, Shigella boydii CDC 3083-94
MVDMKTTHTSLPFAGHTLHFVEFDPANFCEQDLLWLPHYAQLQHAGRKRKAEHLAGRIAAIYALREYGYKCVPAIGELRQPVWPAEVYGSISHCGATALAVVSRQPIGVDIEEIFSAQTATELTDNIITPAEHERLADCGLAFSLALTLAFSAKESAFKASEIQTDAGFLDYQIISWNKQQVIIHRENEMFAVHWQIKEKIAITLCQHD

SEQ ID NO: 8 - protein, 209 aa, Escherichia coli IAI39
MVDMKTTHTALPFTGHTLHFVEFDPASFREQDLLWLPHYAQLQHAGRKRKTEHLAGRIAAVYALREYGYKCVPAIGELRQPVWPAGVYGSISHCGTTALAVVSRQPIGIDIEEIFSVQTARELTDNIITPAEHERLAECGLTFSLALTLAFSAKESAFKASKIQAAQGFLDYQIISWNKQRIIIHRENEMFAVHWQIKEKIVITLCQHD

SEQ ID NO: 9 - protein, 209 aa, Escherichia coli 536
MVDMKTTHTSLPFAGHTLHFVEFDPASFREQDLLWLPHYAQLQHAGRKRKTEHLAGRIAAIYALREYGYKCVPAIGELRQPVWPAGVYGSISHCGTTALAVVSRQPIGIDIEEIFSAQTARELTDNIITPAEHKRLADCGLAFPLALTLAFSAKESAFKASEIQAAQGFLDYQIISWNKQQIIIRLEDEQFAVHWQIKEKIVITLCQHD

SEQ ID NO: 10 - protein, 256 aa, Escherichia coli UMN026
MRVVHAGCGVNALSGLQKSCQFNILQDHVGLISVAHQAVLRLSSVSNIVDMKTTHTALPFAGHTLHFVEFDPASFREQDLLWLPHYAQLQHAGRKRKTEHLAGRIAAVYALREYGYKYVPAIGELRQPVWPAEVYGSISHCGTTALAVVSRQPIGIDIEEIFSVQTARELTDNIITPAEHERLAECGLTFSLALTLAFSAKESAFKASKIQAAQGFLDYQIISWNKQRIIIRLEDEQFAVHWQIKEKIVITLCQHD

SEQ ID NO: 11 - protein, 1168 aa, Mycobacterium smegmatis MC2 155
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

SEQ ID NO: 12 - protein, 1173 aa, Mycobacterium smegmatis MC2 155
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 13 - protein, 1168 aa, Mycobacterium tuberculosis H37Rv
MSINDQRLTRRVEDLYASDAQFAAASPNEAITQAIDQPGVALPQLIRMVMEGYADRPALGQRALRFVTDPDSGRTMVELLPRFETITYRELWARAGTLATALSAEPAIRPGDRVCVLGFNSVDYTTIDIALIRLGAVSVPLQTSAPVTGLRPIVTETEPTMIATSIDNLGDAVEVLAGHAPARLVVFDYHGKVDTHREAVEAARARLAGSVTIDTLAELIERGRALPATPIADSADDALALLIYTSGSTGAPKGAMYRESQVMSFWRKSSGWFEPSGYPSITLNFMPMSHVGGRQVLYGTLSNGGTAYFVAKSDLSTLFEDLALVRPTELCFVPRIWDMVFAEFHSEVDRRLVDGADRAALEAQVKAELRENVLGGRFVMALTGSAPISAEMTAWVESLLADVHLVEGYGSTEAGMVLNDGMVRRPAVIDYKLVDVPELGYFGTDQPYPRGELLVKTQTMFPGYYQRPDVTAEVFDPDGFYRTGDIMAKVGPDQFVYLDRRNNVLKLSQGEFIAVSKLEAVFGDSPLVRQIFIYGNSARAYPLAVVVPSGDALSRHGIENLKPVISESLQEVARAAGLQSYEIPRDFIIETTPFTLENGLLTGIRKLARPQLKKFYGERLERLYTELADSQSNELRELRQSGPDAPVLPTLCRAAAALLGSTAADVRPDAHFADLGGDSLSALSLANLLHEIFGVDVPVGVIVSPASDLRALADHIEAARTGVRRPSFASIHGRSATEVHASDLTLDKFIDAATLAAAPNLPAPSAQVRTVLLTGATGFLGRYLALEWLDRMDLVNGKLICLVRARSDEEAQARLDATFDSGDPYLVRHYRELGAGRLEVLAGDKGEADLGLDRVTWQRLADTVDLIVDPAALVNHVLPYSQLFGPNAAGTAELLRLALTGKRKPYIYTSTIAVGEQIPPEAFTEDADIRAISPTRRIDDSYANGYANSKWAGEVLLREAHEQCGLPVTVFRCDMILADTSYTGQLNLPDMFTRLMLSLAATGIAPGSFYELDAHGNRQRAHYDGLPVEFVAEAICTLGTHSPDRFVTYHVMNPYDDGIGLDEFVDWLNSPTSGSGCTIQRIADYGEWLQRFETSLRALPDRQRHASLLPLLHNYREPAKPICGSIAPTDQFRAAVQEAKIGPDKDIPHLTAAIIAKYISNLRLLGLL

SEQ ID NO: 14 - protein, 1174 aa, Nocardia iowensis NRRL 5646
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 15 - protein, 1174 aa, Mycobacterium sp. JLS
MSTETREARLQQRIAHLFATDPQFAAARPDPRISDAVDRDDARLTAIVSAVMSGYADRPALGQRAAEFATDPQTGRTTMELLPRFDTITYRELLDRVRALTNAWHADGVRPGDRVAILGFTGIDYTVVDLALIQLGAVAVPLQTSAAVEALRPIVAETEPMLIATGVDHVDAAAELALTGHRPSQVVVFDHREQVDDERDAVRAATARLGDAVPVETLAEVLRRGAHLPAVAPHVFDEADPLRLLIYTSGSTGAPKGAMYPESKVAGMWRASAKAAWNNDQTAIPSITLNFLPMSHVMGRGLLCGTLSTGGTAYFAARSDLSTLLEDLRLVRPTQLSFVPRIWDMLFQEFVGEVDRRVNDGADRPTAEADVLAELRQELLGGRFVTAMTGSAPISPEMKTWVETLLDMHLVEGYGSTEAGAVFVDGHIQRPPVLDYKLVDVPDLGYFSTDRPHPRGELLVRSTQLFPGYYKRPDVTAEVFDDDGFYRTGDIVAELGPDQLQYLDRRNNVLKLAQGEFVTISKLEAVFAGSALVRQIFVYGNSARSYLLAVVVPTDDAVARHDPASLKTAISASLQQAAKTAGLQSYELPRDFLVETQPFTLENGLLTGIRKLARPKLKARYGDRLEALYVELAEGQAGELRTLRRDGAKRPVAETVGRAAAALLGAAAADVRPDAHFTDLGGDSLSALTFGNLLQEIFGVDVPVGVIVSPAADLASIAAYIETEQASTGKRPTYASVHGRDAEQVRARDLTLDKFIDAETLSAATELPVPIGEVRTVLLTGATGFLGRYLALDWLERMALVDGKVICLVRAKDDAAARKRLDDTFDSGDPKLLAHYRKLAADHLEVLAGDKGEADLGLPHQVWQRLADTVDLIVDPAALVNHVLPYSQLFGPNALGTAELIRLALTTRIKPFTYVSTIGVGAGIEPGRFTEDDDIRVISPTRAVDTGYANGYGNSKWAGEVLLREAHDLCGLPVAVFRCDMILADTTYAGQLNLPDMFTRMMVSLVTTGIAPKSFHPLDAKGHRQRAHYDGLPVEFVAESISALGAQAVDEAGTGFATYHVMNPHDDGIGLDEFVDWLVEAGYRIDRIDDYAAWLQRFETALRALPERTRQYSLLPLLHNYQRPAHPINGAMAPTDRFRAAVQEAKLGPDKDIPHVTPGVIVKYATDLELLGLI

SEQ ID NO: 16 - protein, 1148 aa, Streptomyces griseus
MAEPLDAATASAHDPGQGLAEALAAVEPGRALAEVMASVLEGHGDRPALGERAREPETGRLLPHFDTISYRELWSRVRALAGRWHHDPEYPLGPGDRICTLGFTSTDYATLDLACIHLGAVPVPLPSNAPLPRLAPVVEESGPTVLAASVDRLDTAIDVVLASSTIRRLLVFDDGPGATRPGGALAAARQRLSGSPVTVDTLAGLIDRGRDLPPPPLYIPDPGEDPLALLIYTSGSTGAPKGAMYTQRLLGTAWYGFSYGAADTPAISVLYLPQSHLAGRYAVMGSLVKGGTGYFTAADDLSTLFEDIALVRPTELTMVPRLCDMLLQHYRSERDRRADEPGDIEAAVTKAVREDFLGGRVAKAFVGTAPLSAELTAFVESVLGFHLYTGYGSTEAGGVLLDTVVQRPPVTDYKLVDVPELGYYATDLPHPRGELLLKSHTLIPGYYRRPDLTAAIFDADGYYRTGDVFAETGPDRLVYVDRTKDTLKLSQGEFVAVSRLETVLLDSPLVQHLYLYGNSERAYLLAVVVPTPDALAGCGGDTEALRPLLMESLRSVARRAGLNAYEIPRGILVEPEPFSPENGLFTESHKLLRPRLKERYGPALELLYDRLADGQDRRLRELRRTGADRPVQETVLRAAQALLGSPGSDLRPGAHFTDLGGDSLSAVSFSELMKEIFHVDVPVGAIIGPAADLAEVARYITAARRPAGAPRPTPASVHGEHRTEVRAGDLAPEKFLDAPTLAAAPALPRPDGDVRTVLLTGATGYLGRFLCLEWLERLAPSGGRLVCLVRGSDATVAARRLEAAFDSGDTALLRRYRKAAGKTLDVVAGDIGEPLLGLAEETWRELAGAVDLIVHPAALVNHLLPYGELFGPNVVGTAEAIRLALTTRLKPVNHVSTVAVCLGTPAETADENADIRAAVPVRTTGQGYADGYATSKWAGEVLLREAHERYGLPVAVFRSDMVLAHRTYTGQVNVPDVLTRLLLSLVATGIAPGSFYRTDTRAHYDGLPVDFTAEAVVALGAPITEGHRTFNVLNPHDDGVSLDTFVDWLIEAGHPIRRIDDHGAWLTRFTAALRALPEKQRQHSLLPLIGAWAEPGEGAPGPLLPARRFHAAVRAAGVGPERDIPRVSPDLIRKYVTDLRALGLLAGP

SEQ ID NO: 17 - protein, 224 aa, Bacillus subtilis ATCC 21332
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 18 - protein, 222 aa, Mycobacterium smegmatis MC155
MGTDSLLSLVLPDRVASAEVYDDPPGLSPLPEEEPLIARSVAKRRNEFVTVRYCARQALGELGVGPVPILKGDKGEPCWPDGVVGSLTHCQGFRGAVVGRSTDVRSVGIDAEPHDVLPNGVLDAITLPIERAELRGLPGDLHWDRILFCAKEATYKAWYPLTHRWLGFEDAHITFEVDGSGTAGSFRSRILIDPVAEHGPPLTALDGRWSVRDGLAVTAIVL

SEQ ID NO: 19 - protein, 242 aa, Pseudomonas aeruginosa
MRAMNDRLPSFCTPLDDRWPLPVALPGVQLRSTRFDPALLQPGDFALAGIQPPANILRAVAKRQAEFLAGRLCARAALFALDGRAQTPAVGEDRAPVWPAAISGSITHGDRWAAALVAARGDWRGLGLDVETLLEAERARYLHGEILTEGERLRFADDLERRTGLLVTLAFSLKESLFKALYPLVGKRFYFEHAELLEWRADGQARLRLLTDLSPEWRHGSELDAQFAVLDGRLLSLVAVGA

SEQ ID NO: 20 - DNA, 70 bp, Artificial Sequence
  furF primer
gcaggttggcttttctcgttcaggctggcttatttgccttcgtgcgcatgattccggggatccgtcgacc

SEQ ID NO: 21 - DNA, 69 bp, Artificial Sequence
  furR primer
cacttcttctaatgaagtgaaccgcttagtaacaggacagattccgcatgtgtaggctggagctgcttc

SEQ ID NO: 22 - DNA, 24 bp, Artificial Sequence
  furVF primer
attgaagcctgccagagcgtgtta

SEQ ID NO: 23 - DNA, 24 bp, Artificial Sequence
  furVR primer
cctgatgtgatgcggcgtagactc

SEQ ID NO: 24 - DNA, 45 bp, Artificial Sequence
  EntD-for primer
caggaggaattcaccatggtcgatatgaaaactacgcatacctcc

SEQ ID NO: 25 - DNA, 42 bp, Artificial Sequence
  EntD-rev primer
agatgtaagcttttaatcgtgttggcacagcgttatgactat

SEQ ID NO: 26 - DNA, 3167 bp, Artificial Sequence
  pMA_1001546 plasmid
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgccatgagctcaggcacctgctttacactttcgcccgtggtcagtgatggctgcgggcgaatcgtaccagatgttgtcaactattataaaagctcttcgtacgagaccattgtgatatcctcggggaaatcagggtgtgcggcgcatacagccattttgtagccgggatcgacctcatacgttttgatatagcatggggaatggctgtccggaagctcaatggatacttgtccgtcctgatgcaggcgcactgaaaaggaatcaagcggaagcgataagcctttgccttcctgtttgataaagctttctttcattgaccatagatgataaaaatagtctgtctgctcgtccttgtcttttgctaaaaggtcgctgtactctgtttttgaaaagaagcgcttggcgatctcaaggctgatcggtttcgttttttcgatatctatgccgatcggctgtgaatcaaacgcaccaatgacccagcggccggagtgagaaatgttgaaatgagcgtcgggaagatcagggatgcacggcttcccgtattcctgcgtgctaaagcggatatcggatttgtccaactgatactgcctgcttatgactgagcgaacgagcacatctcccagcagggtgcggtgagcatcttctttatgataaaatctccggcatttctcccgtttttcaggtgatatgaaagtcatgaaccgttcattttcttcctgtgaaagcgggcggtccatataaattccgtaaatcttcatggtttattcctccttaaaacgcaaaactgcctgatgcgctacgcttatcaggtacctcttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 27 - DNA, 7998 bp, Artificial Sequence
  pDF14 plasmid
ggggaattgtgagcggataacaattcccctgtagaaataattttgtttaactttaataaggagatataccatgggcaccgatagcctgttgagcttggtgctgccggaccgcgtcgcgtctgcggaagtgtatgacgatcctccgggcctgtctcctctgccggaggaggaaccgctgatcgcacgttctgttgccaagcgccgtaatgagttcgtcaccgtgcgctattgcgcgcgtcaagcgctgggtgaactgggtgttggcccggtcccgatcctgaagggtgataaaggtgaaccgtgctggccggacggtgtcgtcggtagcctgacccactgtcagggtttccgtggtgcggtcgttggtcgttccaccgatgtccgcagcgttggtatcgatgccgaaccgcatgatgtgttgccgaacggcgttctggatgcaattaccctgccaattgagcgcgcggaactgcgcggtctgccgggcgatctgcactgggaccgcatcctgttctgtgcgaaggaagctacctacaaagcctggtacccgctgacccaccgctggctgggctttgaagatgcgcacattacctttgaggtcgatggtagcggcacggcgggcagctttcgttctcgtattctgatcgacccggttgcggaacatggtccgccgctgaccgctctggacggtcgctggagcgtccgtgatggtctggcggtgaccgcgattgtcctgtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttaagaattccctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 28 - DNA, 3543 bp, Artificial Sequence
  pJ204_38022 plasmid
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttcgctgaatgataagcgtagcgcatcaggcagttttgcgttttaaggaggaataaaccatgcgcgcgatgaacgacagactgccgagcttttgcaccccgctggacgatcgttggcctctgccggtcgccctgccgggtgtccaattgcgcagcacgcgtttcgacccggcgttgctgcaaccgggtgactttgcattggcgggcattcagcctccggcaaatatcctccgtgcggttgcaaagcgtcaagcggagtttttggccggtcgtctgtgtgcgcgtgcggctctgttcgccctggacggccgtgcgcagaccccggcagttggtgaggatcgcgcaccggtgtggccagcggcgatcagcggtagcatcacgcatggcgaccgttgggcggcagcgctggtggcagctcgcggtgattggcgtggcctgggcctggatgtcgaaacgttgctggaagcggaacgtgcccgctacctgcatggcgagattttgaccgagggcgaacgcttgcgtttcgccgatgatctggaacgtcgcaccggtttactggttacgctggcgttttccctgaaagaaagcctgtttaaagcactgtacccgctggtgggtaagcgcttctatttcgaacacgcggagctgctggagtggcgtgcagatggccaggcgcgtctgcgcctgctgaccgatctgagcccggaatggcgccacggctcggagctggacgctcagttcgctgttttggacggtcgcttgctgagcctggtggctgttggtgcgtagttgacaacatctggtacgattcgcccgcagccatcactgaccacgggcgaaagtgtaaagcaggtgcctcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 29 - DNA, 71 bp, Artificial Sequence
  cat-for primer
agccgggacgtacgtggtatatgagcgtaaacacccacttctgatgctaagtgtaggctggagctgcttcg

SEQ ID NO: 30 - DNA, 127 bp, Artificial Sequence
  cat-rev primer
attcgagactgatgacaaacgcaaaactgcctgatgcgctacgcttatcattgaatctattatacagaaaaattttcctgaaagcaaataaattttttatgattgacatgggaattagccatggtcc

SEQ ID NO: 31 - DNA, 88 bp, Artificial Sequence
  sfp-for primer
tgataagcgtagcgcatcaggcagttttgcgtttgtcatcagtctcgaatatgaagatttacggaatttatatggaccgcccgctttc

SEQ ID NO: 32 - DNA, 26 bp, Artificial Sequence
  sfp-rev primer
aggcacctgctttacactttcgcccg

SEQ ID NO: 33 - DNA, 61 bp, Artificial Sequence
  pptmc155-for primer
gcatcaggcagttttgcgtttgtcatcagtctcgaatatgggcaccgatagcctgttgagc

SEQ ID NO: 34 - DNA, 72 bp, Artificial Sequence
  pptmc155-rev primer
tcgcccgtggtcagtgatggctgcgggcgaatcgtaccagatgttgtcaattacaggacaatcgcggtcacc

SEQ ID NO: 35 - DNA, 75 bp, Artificial Sequence
  pcpS-for primer
tgataagcgtagcgcatcaggcagttttgcgtttgtcatcagtctcgaatatgcgcgcgatgaacgacagactgc

SEQ ID NO: 36 - DNA, 26 bp, Artificial Sequence
  pcpS-rev primer
aggcacctgctttacactttcgcccg

SEQ ID NO: 37 - DNA, 27 bp, Artificial Sequence
  sfpSOE-for primer
agccgggacgtacgtggtatatgagcg

SEQ ID NO: 38 - DNA, 26 bp, Artificial Sequence
  sfpSOE-rev primer
aggcacctgctttacactttcgcccg

SEQ ID NO: 39 - DNA, 27 bp, Artificial Sequence
  pptmc155SOE-for primer
agccgggacgtacgtggtatatgagcg

SEQ ID NO: 40 - DNA, 23 bp, Artificial Sequence
  pptmc155SOE-rev primer
tcgcccgtggtcagtgatggctg

SEQ ID NO: 41 - DNA, 27 bp, Artificial Sequence
  pcpSSOE-for primer
agccgggacgtacgtggtatatgagcg

SEQ ID NO: 42 - DNA, 26 bp, Artificial Sequence
  pcpSSOE-rev primer
aggcacctgctttacactttcgcccg

SEQ ID NO: 43 - DNA, 71 bp, Artificial Sequence
  deltaentDcat-for primer
tgataagcgtagcgcatcaggcagttttgcgtttgtcatcagtctcgaatgtgtaggctggagctgcttcg

SEQ ID NO: 44 - DNA, 73 bp, Artificial Sequence
  deltaentDcat-rev primer
tcgcccgtggtcagtgatggctgcgggcgaatcgtaccagatgttgtcaagacatgggaattagccatggtcc

SEQ ID NO: 45 - DNA, 23 bp, Artificial Sequence
  screening-for
ggcaagcagcagccgaagaagta

SEQ ID NO: 46 - DNA, 25 bp, Artificial Sequence
  screening-rev
ggtggccattcgtgggacagtatcc

SEQ ID NO: 47 - DNA, 12397 bp, Artificial Sequence
  p7P36 plasmid
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttagaattccataattgctgttaggagatatatatggcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggctgtgacggtttgcgtggttttcagccacagcaaaccgagcaaacgctgcgccagattttgcaggatgtcaaagccgccaacgctcttccattgttaatgcaaatacgtctgccttacaactatggtcgtcgttataatgaagcctttagcgccatttaccccaaactcgccaaagagtttgatgttccgctgctgcccttttttatggaagaggtctgcctcaagccacaatggatgcaggatgacggtattcatcccaaccgcgacgcccagccgtttattgccgactggatggcgaagcagttgcagcctttaaccaatcatgactcataagcttctaaggaaataataggagattgaaaatggcaacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagctatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaacctagggataatagaggttaagagcggccagatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaaactttatcgaaatggccatccattcttggtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

(19) United States
(12) Patent Application Publication
(10) Pub. No.: US 2017/0096688 A1
(43) Pub. Date: Apr. 6, 2017

(54) METHODS AND COMPOSITIONS FOR ENHANCED PRODUCTION OF FATTY ALDEHYDES AND FATTY ALCOHOLS

(71) Applicant: REG LIFE SCIENCES, LLC, Ames, IA (US)

(72) Inventors: Zhihao HU, South San Francisco, CA (US); Derek L. GREENFIELD, South San Francisco, CA (US); Vikranth Arlagadda, South San Francisco, CA (US)

(21) Appl. No.: 15/155,856

(22) Filed: May 16, 2016

Related U.S. Application Data
(63) Continuation of application No. 14/179,944, filed on Feb. 13, 2014, now abandoned.
Application No. 14/179,944 is a division of application No. 13/359,127, filed on Jan. 26, 2012, now abandoned.
(60) Provisional application No. 61/436,542, filed on Jan. 26, 2011.

(51) Int. Cl. C12P 7/24; C12N 9/12; C12N 15/70; C12P 7/04

(52) U.S. Cl. CPC C12P 7/24; C12P 7/04; C12N 9/1288; C12Y 207/08007; C12N 15/70

(57) ABSTRACT

The invention relates to the use of EntD polypeptides, polynucleotides encoding the same, and homologues thereof to enhance the production of fatty aldehydes and fatty alcohols in a host cell.

### CROSS-REFERENCE TO RELATED APPLICATIONS

This application is a divisional of U.S. patent application Ser. No. 13/359,127, filed Jan. 26, 2012 and which claimed the benefit of priority of U.S. Provisional Application Ser. No. 61/436,542, filed Jan. 26, 2011, all of which applications are expressly incorporated herein by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 126,717 Byte ASCII (Text) file named “707360_ST25.TXT,” created on Jan. 26, 2011. It is understood that the Patent and Trademark Office will make the necessary changes in application number and filing date for the instant application.

### BACKGROUND OF THE INVENTION

Crude petroleum is a limited, natural resource found in the Earth in liquid, gaseous, and solid forms. Although crude petroleum is a valuable resource, it is discovered and extracted from the Earth at considerable financial and environmental costs. Moreover, in its natural form, crude petroleum extracted from the Earth has few commercial uses. Crude petroleum is a mixture of hydrocarbons (e.g., paraffins (or alkanes), olefins (or alkenes), alkynes, napthenes (or cycloalkanes), aliphatic compounds, aromatic compounds, etc.) of varying length and complexity. In addition, crude petroleum contains other organic compounds (e.g., organic compounds containing nitrogen, oxygen, sulfur, etc.) and impurities (e.g., sulfur, salt, acid, metals, etc.). Hence, crude petroleum must be refined and purified at considerable cost before it can be used commercially.

Crude petroleum is also a primary source of raw materials for producing petrochemicals. The two main classes of raw materials derived from petroleum are short chain olefins (e.g., ethylene and propylene) and aromatics (e.g., benzene and xylene isomers). These raw materials are derived from longer chain hydrocarbons in crude petroleum by cracking it at considerable expense using a variety of methods, such as catalytic cracking, steam cracking, or catalytic reforming. These raw materials can be used to make petrochemicals such as monomers, solvents, detergents, and adhesives, which otherwise cannot be directly refined from crude petroleum.

Petrochemicals, in turn, can be used to make specialty chemicals, such as plastics, resins, fibers, elastomers, pharmaceuticals, lubricants, and gels. Particular specialty chemicals that can be produced from petrochemical raw materials include fatty acids, hydrocarbons (e.g., long chain, branched chain, saturated, unsaturated, etc.), fatty aldehydes, fatty alcohols, esters, ketones, lubricants, etc.

Due to the inherent challenges posed by petroleum, there is a need for a renewable petroleum source that does not need to be explored, extracted, transported over long distances, or substantially refined like crude petroleum. There is also a need for a renewable petroleum source which can be produced economically without creating the type of environmental damage produced by the petroleum industry and the burning of petroleum-based fuels. For similar reasons, there is also a need for a renewable source of chemicals which are typically derived from petroleum.

One method of producing renewable petroleum is by engineering microorganisms to produce renewable petroleum products. Some microorganisms have long been known to possess a natural ability to produce petroleum products (e.g., yeast to produce ethanol). More recently, the development of advanced biotechnologies has made it possible to metabolically engineer an organism to produce bioproducts and biofuels. Bioproducts (e.g., chemicals) and biofuels (e.g., biodiesel) are renewable alternatives to petroleum-based chemicals and fuels, respectively. Bioproducts and biofuels can be derived from renewable sources, such as plant matter, animal matter, and organic waste matter, which are collectively known as biomass.

Biofuels can be substituted for any petroleum-based fuel (e.g., gasoline, diesel, aviation fuel, heating oil, etc.), and offer several advantages over petroleum-based fuels. Biofuels do not require expensive and risky exploration or extraction. Biofuels can be produced locally and therefore do not require transportation over long distances. In addition, biofuels can be made directly and require little or no additional refining. Furthermore, the combustion of biofuels causes less of a burden on the environment since the amount of harmful emissions (e.g., green house gases, air pollution, etc.) released during combustion is reduced as compared to the combustion of petroleum-based fuels. Moreover, biofuels maintain a balanced carbon cycle because biofuels are produced from biomass, a renewable, natural resource. Although combustion of biofuels releases carbon (e.g., as carbon dioxide), this carbon will be recycled during the production of biomass (e.g., the cultivation of crops), thereby balancing the carbon cycle, which is not achieved with the use of petroleum based fuels.

Biologically derived chemicals offer similar advantages over petrochemicals that biofuels offer over petroleum-based fuels. In particular, biologically derived chemicals can be converted from biomass to the desired chemical product directly without extensive refining, unlike petrochemicals, which must be produced by refining crude petroleum to recover raw materials which are then processed further into the desired petrochemical.

Aldehydes are used to produce many specialty chemicals. For example, aldehydes are used to produce polymers, resins (e.g., Bakelite), dyes, flavorings, plasticizers, perfumes, pharmaceuticals, and other chemicals, some of which may be used as solvents, preservatives, or disinfectants. In addition, certain natural and synthetic compounds, such as vitamins and hormones, are aldehydes, and many sugars contain aldehyde groups. Fatty aldehydes can be converted to fatty alcohols by chemical or enzymatic reduction.

Fatty alcohols have many commercial uses. Worldwide annual sales of fatty alcohols and their derivatives are in excess of U.S. $1 billion. The shorter chain fatty alcohols are used in the cosmetic and food industries as emulsifiers, emollients, and thickeners. Due to their amphiphilic nature, fatty alcohols behave as nonionic surfactants, which are useful in personal care and household products, such as, for example, detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats.

Carboxylic acid reductase (CAR) is an enzyme cloned from Nocardia sp. strain NRRL 5646 that has been demonstrated to catalyze the reduction of aryl carboxylic acids to aldehydes and alcohols in an ATP-, NADPH-, and Mg²⁺-dependent manner (Li et al., J. Bacteriol., 179(11): 3482-3487 (1997); He et al., Appl. Environ. Microbiol., 70(3): 1874-1881 (2004)). Basic Local Alignment Search Tool (BLAST) analysis has led to the identification of CAR homologues in numerous microorganisms (He et al., supra; U.S. Pat. No. 7,425,433; and International Patent Application Publication No. WO 2010/062480). It was recently demonstrated that co-expression of a gene encoding any one of three CAR homologues, i.e., CarA or CarB from Mycobacterium smegmatis or FadD9 from Mycobacterium tuberculosis, along with a gene encoding a thioesterase (i.e., ‘tesA) in Escherichia coli cultured in a medium containing fatty acids results in high titers of fatty alcohol production and detectable levels of fatty aldehyde production (International Patent Application Publication No. WO 2010/062480).

BLAST analysis demonstrated that Nocardia CAR contains an N-terminal domain with high homology to AMP-binding proteins and a C-terminal domain with high homology to NADPH binding proteins (He et al., supra). Nocardia CAR and several of its homologues contain a putative attachment site for 4′-phosphopantetheine (PPT) (He et al., supra, and U.S. Pat. No. 7,425,433), which is a prosthetic group derived from Coenzyme A. Subsequently, it was demonstrated that recombinant Nocardia phosphopantetheine transferase (PPTase) can catalyze the incorporation of a radiolabeled PPT moiety into a recombinant CAR substrate, and that co-expression of Nocardia CAR and Nocardia PPTase in E. coli results in an increased level of vanillic acid reduction as compared to the level of vanillic acid reduction observed in E. coli expressing Nocardia CAR in the absence of Nocardia PPTase (Venkitasubramanian et al., J. Biol. Chem., 282(1): 478-485 (2007)).

PPTases are known to display varying substrate spectrums (Lambalot et al., Chem. Biol., 3: 923-936 (1996)). For example, Bacillus subtilis is known to contain two PPTases, namely AcpS and Sfp. It has been demonstrated that AcpS selectively recognizes acyl carrier protein (ACP) and D-alanyl carrier protein (DCP) of primary metabolism as substrates, whereas Sfp recognizes more than forty ACPs and peptidyl carrier proteins (PCP) of secondary metabolism as substrates (Mootz et al., J. Biol. Chem., 276 (40): 37289-37298 (2001)).

E. coli is known to contain three PPTases, namely, AcpS, AcpT, and EntD. It has been demonstrated that AcpS and AcpT specifically transfer PPT to ACP, whereas EntD transfers PPT to the EntB and EntF members of the Ent biosynthetic gene cluster responsible for producing the iron scavenging enterobactin siderophore (Lambalot et al., supra, and Flugel et al., J. Biol. Chem., 276(40): 37289-37298 (2001)). In heterologous expression systems, selection of an appropriate PPTase for a given substrate is an important consideration due, in part, to the narrow substrate specificities of many PPTases (Pfeifer et al., Microbiol. Mol. Biol. Rev., 65(1): 106-118 (2001)).

There remains a need for methods and compositions for enhancing the production of biologically derived chemicals, such as fatty aldehydes and fatty alcohols. This invention provides such methods and compositions. The invention further provides products derived from the fatty aldehydes and fatty alcohols produced by the methods described herein, such as fuels, surfactants, and detergents.

### BRIEF SUMMARY OF THE INVENTION

The invention provides improved methods of producing a fatty aldehyde or a fatty alcohol in a host cell. In one embodiment, the method comprises (a) expressing a polynucleotide sequence encoding a PPTase comprising an amino acid sequence having at least 80% identity to the amino acid sequence of SEQ ID NO: 1 in the host cell, (b) culturing the host cell expressing the PPTase in a culture medium under conditions permissive for the production of a fatty aldehyde or a fatty alcohol, and (c) isolating the fatty aldehyde or fatty alcohol from the host cell.

In another embodiment, the method comprises (a) providing a vector comprising a polynucleotide sequence having at least 80% identity to the polynucleotide sequence of SEQ ID NO: 2 to the host cell, (b) culturing the host cell under conditions in which the polynucleotide sequence of the vector is expressed to produce a polypeptide that results in the production of a fatty aldehyde or a fatty alcohol, and (c) isolating the fatty aldehyde or fatty alcohol from the host cell.

The invention also provides a recombinant host cell comprising (a) a polynucleotide sequence encoding a PPTase comprising an amino acid sequence having at least 80% identity to the amino acid sequence of SEQ ID NO: 1 and (b) a polynucleotide encoding a polypeptide having carboxylic acid reductase activity, wherein the recombinant host cell is capable of producing a fatty aldehyde or a fatty alcohol.

In another embodiment, the recombinant host cell comprises (a) a polynucleotide sequence having at least 80% identity to the polynucleotide sequence of SEQ ID NO: 2 and (b) a polynucleotide encoding a polypeptide having carboxylic acid reductase activity, wherein the recombinant host cell is capable of producing a fatty aldehyde or a fatty alcohol.

In the aforementioned embodiments of the invention wherein the polynucleotide sequence encodes an endogenous PPTase, the endogenous PPTase is overexpressed.

The invention also provides a method of producing a fatty aldehyde or a fatty alcohol in a host cell, which comprises increasing the level of expression and/or activity of an endogenous PPTase comprising an amino acid sequence having at least 80% identity to the amino acid sequence of SEQ ID NO: 1 in the host cell as compared to the level of expression and/or activity of the PPTase in a corresponding wild-type host cell, (b) culturing the host cell expressing the PPTase in a culture medium under conditions permissive for the production of a fatty aldehyde or a fatty alcohol, and (c) isolating the fatty aldehyde or fatty alcohol from the host cell.

Further provided are methods of improving the production of a fatty aldehyde or a fatty alcohol in a host cell cultured in a medium containing iron. In one embodiment, the invention provides a method for increasing the production of fatty aldehyde or fatty alcohol production in a host cell whose production of fatty aldehyde or fatty alcohol is sensitive to the amount of iron present in a medium for the host cell. The method comprises (a) expressing a polynucleotide sequence encoding a PPTase in the host cell, (b) culturing the host cell expressing the PPTase in a medium containing iron under conditions permissive for the production of a fatty aldehyde or a fatty alcohol, and (c) isolating the fatty aldehyde or fatty alcohol from the host cell. As a result of this method, expression of the PPTase results in an increase in the production of fatty aldehyde or fatty alcohol in the host cell as compared to the production of fatty aldehyde or fatty alcohol under the same conditions in the same host cell except for not expressing the PPTase.

The invention also provides a method for relieving iron-induced inhibition of fatty aldehyde or fatty alcohol production in a host cell whose production of fatty aldehyde or fatty alcohol is sensitive to the amount of iron present in a medium for the host cell. The method comprises (a) expressing a polynucleotide sequence encoding a PPTase in the host cell and (b) culturing the host cell expressing the PPTase in a medium containing iron under conditions permissive for the production of a fatty aldehyde or a fatty alcohol. As a result of this method, expression of the PPTase causes an increase in the production of fatty aldehyde or fatty alcohol in the host cell as compared to the production of fatty aldehyde or fatty alcohol under the same conditions in the same host cell except for not expressing the PPTase.

Further provided is a method for relieving iron-induced inhibition of a polypeptide having carboxylic acid reductase activity in a host cell whose activity is sensitive to the amount of iron present in a medium for the host cell. The method comprises (a) expressing a polynucleotide sequence encoding a phosphopanthetheinyl transferase (PPTase) in the host cell, and (b) culturing the host cell expressing said PPTase in a medium containing iron. As a result of this method, the activity of a polypeptide having carboxylic acid reductase activity is increased upon expression of the PPTase as compared to the activity of the polypeptide having carboxylic acid reductase activity under the same conditions in the same host cell except for not expressing said PPTase.

The invention also provides a method for transferring PPT to a substrate having carboxylic acid reductase activity. The method comprises incubating a PPTase polypeptide comprising an amino acid sequence having at least 80% sequence identity to the amino acid sequence of SEQ ID NO: 1 with said substrate under conditions suitable for transfer of PPT, thereby transferring PPT to the substrate having carboxylic acid reductase activity.

### BRIEF DESCRIPTION OF THE DRAWINGS

FIG. 1 is a line graph of combined fatty aldehyde and fatty alcohol production as assessed by gas chromatography-mass spectroscopy (GC-MS) in a control E. coli strain (DV2) or an E. coli DV2 strain containing a deletion of the fur gene (ALC2) grown in V9-B medium with or without 50 mg/L iron at several time points following induction of fatty aldehyde and fatty alcohol production by the addition of isopropyl-β-D-thiogalactopyranoside (IPTG) to the culture medium.

FIG. 2 is a graph of combined fatty aldehyde and fatty alcohol production as assessed by GC-MS in a control E. coli strain (DV2) or an E. coli DV2 strain containing a deletion of the fur gene (ALC2) grown in V9-B medium in the presence of iron at the indicated concentrations. The bars represent combined fatty aldehyde and fatty alcohol titers, and the line represents the amount of fatty aldehyde and fatty alcohol production relative to the amount of fatty aldehyde and fatty alcohol production in the control DV2 strain cultured in the absence of iron.

FIG. 3 is a bar graph of fatty aldehyde and fatty alcohol production as assessed by GC-MS in E. coli DV2 strains transformed with a control pBAD24 empty vector or a pBAD24 vector expressing the entD gene under the control of an inducible arabinose promoter.

FIG. 4 is a bar graph of fatty alcohol production as assessed by GC-MS in a control E. coli strain not expressing exogenous PPTase or in E. coli strains overexpressing the indicated PPTase.

FIGS. 5A and 5B are images of Coomassie blue-stained gels following sodium dodecyl sulfate polyacrylamide gel electrophoresis (SDS-PAGE) of the indicated samples. In FIG. 5A, lane 1 contains a molecular weight standard, and lane 2 contains recombinant CarB purified from E. coli. In FIG. 5B, recombinant CarB purified from E. coli overexpressing entD (CarB+EntD) and recombinant CarB purified from E. coli in which the entD has been deleted (CarB-EntD) are compared.

FIG. 6 is a bar graph depicting the enzyme activity of recombinant CarB purified from E. coli in which the entD has been deleted (CarB-EntD) as compared to the enzyme activity of recombinant CarB purified from E. coli overexpressing entD (CarB+EntD) as assessed by an in vitro CAR assay.

### DETAILED DESCRIPTION OF THE INVENTION

The invention is based, at least in part, upon the discovery that EntD expression in a host cell facilitates enhanced production of fatty aldehydes and fatty alcohols by the host cell.

The invention provides improved methods of producing a fatty aldehyde or a fatty alcohol in a host cell. In one embodiment, the method comprises (a) expressing a polynucleotide sequence encoding a PPTase comprising an amino acid sequence having at least 80% identity to the amino acid sequence of SEQ ID NO: 1 in the host cell, (b) culturing the host cell expressing the PPTase in a culture medium under conditions permissive for the production of a fatty aldehyde or a fatty alcohol, and (c) isolating the fatty aldehyde or fatty alcohol from the host cell. In those embodiments of this method wherein the polynucleotide sequence encodes an endogenous PPTase, the endogenous PPTase is overexpressed.

In another embodiment, the method comprises (a) providing a vector comprising a polynucleotide sequence having at least 80% identity to the polynucleotide sequence of SEQ ID NO: 2 to the host cell, (b) culturing the host cell under conditions in which the polynucleotide sequence of the vector is expressed to produce a polypeptide whose expression results in the production of a fatty aldehyde or a fatty alcohol, and (c) isolating the fatty aldehyde or fatty alcohol from the host cell. In those embodiments of this method wherein the polynucleotide sequence encodes an endogenous PPTase, the endogenous PPTase is overexpressed.

In yet another embodiment, the method comprises increasing the level of expression and/or activity of an endogenous PPTase comprising an amino acid sequence having at least 80% identity to the amino acid sequence of SEQ ID NO: 1 in the host cell as compared to the level of expression and/or activity of the PPTase in a corresponding wild-type host cell, (b) culturing the host cell expressing the PPTase in a culture medium under conditions permissive for the production of a fatty aldehyde or a fatty alcohol, and (c) isolating the fatty aldehyde or fatty alcohol from the host cell.

As used herein, “fatty aldehyde” means an aldehyde having the formula RCHO characterized by a carbonyl group (C═O). In some embodiments, the fatty aldehyde is any aldehyde made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty aldehyde is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty aldehyde. In certain embodiments, the fatty aldehyde is a C₆, C₈, C₁₀, C₁₂, C₁₃, C₁₄, C₁₅, C₁₆, C₁₇, or C₁₈ fatty aldehyde.

As used herein, “fatty alcohol” means an alcohol having the formula ROH. In some embodiments, the fatty alcohol is any alcohol made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty alcohol is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty alcohol. In certain embodiments, the fatty alcohol is a C₆, C₈, C₁₀, C₁₂, C₁₃, C₁₄, C₁₅, C₁₆, C₁₇, or C₁₈ fatty alcohol.

The R group of a fatty aldehyde or a fatty alcohol can be a straight chain or a branched chain. Branched chains may have more than one point of branching and may include cyclic branches. In some embodiments, the branched fatty aldehyde or branched fatty alcohol comprises a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ branched fatty aldehyde or branched fatty alcohol. In particular embodiments, the branched fatty aldehyde or branched fatty alcohol is a C₆, C₈, C₁₀, C₁₂, C₁₃, C₁₄, C₁₅, C₁₆, C₁₇, or C₁₈ branched fatty aldehyde or branched fatty alcohol. In certain embodiments, the hydroxyl group of the branched fatty aldehyde or branched fatty alcohol is in the primary (C₁) position.

In certain embodiments, the branched fatty aldehyde or branched fatty alcohol is an iso-fatty aldehyde or iso-fatty alcohol, or an anteiso-fatty aldehyde or anteiso-fatty alcohol. In exemplary embodiments, the branched fatty aldehyde or branched fatty alcohol is selected from iso-C_{7:0}, iso-C_{8:0}, iso-C_{9:0}, iso-C_{10:0}, iso-C_{11:0}, iso-C_{12:0}, iso-C_{13:0}, iso-C_{14:0}, iso-C_{15:0}, iso-C_{16:0}, iso-C_{17:0}, iso-C_{18:0}, iso-C_{19:0}, anteiso-C_{7:0}, anteiso-C_{8:0}, anteiso-C_{9:0}, anteiso-C_{10:0}, anteiso-C_{11:0}, anteiso-C_{12:0}, anteiso-C_{13:0}, anteiso-C_{14:0}, anteiso-C_{15:0}, anteiso-C_{16:0}, anteiso-C_{17:0}, anteiso-C_{18:0}, and anteiso-C_{19:0} branched fatty aldehyde or branched fatty alcohol.

The R group of a branched or unbranched fatty aldehyde or a fatty alcohol can be saturated or unsaturated. If unsaturated, the R group can have one or more than one point of unsaturation. In some embodiments, the unsaturated fatty aldehyde or unsaturated fatty alcohol is a monounsaturated fatty aldehyde or monounsaturated fatty alcohol. In certain embodiments, the unsaturated fatty aldehyde or unsaturated fatty alcohol is a C6:1₉ C7:1₉ C8:1, C9:1, C10:1, C11:1, C12:1, C13:1, C14:1, C15:1, C16:1, C17:1, C18:1, C19:1, C20:1, C21:1, C22:1, C23:1, C24:1, C25:1, or a C26:1 unsaturated fatty aldehyde or unsaturated fatty alcohol. In certain preferred embodiments, the unsaturated fatty aldehyde or unsaturated fatty alcohol is C10:1, C12:1, C14:1, C16:1, or C18:1. In yet other embodiments, the unsaturated fatty aldehyde or unsaturated fatty alcohol is unsaturated at the omega-7 position. In certain embodiments, the unsaturated fatty aldehyde or unsaturated fatty alcohol comprises a cis double bond.

As used herein, the term “fatty acid” means a carboxylic acid having the formula RCOOH. R represents an aliphatic group, preferably an alkyl group. R can comprise between about 4 and about 22 carbon atoms. Fatty acids can be saturated, monounsaturated, or polyunsaturated. In a preferred embodiment, the fatty acid is made from a fatty acid biosynthetic pathway.

As used herein, the term “fatty acid biosynthetic pathway” means a biosynthetic pathway that produces fatty acids. The fatty acid biosynthetic pathway includes fatty acid synthases that can be engineered to produce fatty acids, and in some embodiments can be expressed with additional enzymes to produce fatty acids having desired carbon chain characteristics.

As used herein, the term “fatty acid derivative” means products made in part from the fatty acid biosynthetic pathway of the production host organism. “Fatty acid derivative” also includes products made in part from acyl-ACP or acyl-ACP derivatives. Exemplary fatty acid derivatives include, for example, fatty acids, acyl-CoA, fatty aldehyde, short and long chain alcohols, hydrocarbons, fatty alcohols, and esters (e.g., waxes, fatty acid esters, or fatty esters).

“Polynucleotide” refers to a polymer of DNA or RNA, which can be single-stranded or double-stranded and which can contain non-natural or altered nucleotides. The terms “polynucleotide,” “nucleic acid,” and “nucleic acid molecule” are used herein interchangeably to refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to plasmid, viral, chromosomal, EST, cDNA, mRNA, and rRNA.

The term “nucleotide” as used herein refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like).

The terms “polypeptide” and “protein” refer to a polymer of amino acid residues. The term “recombinant polypeptide” refers to a polypeptide that is produced by recombinant DNA techniques, wherein generally DNA encoding the expressed protein or RNA is inserted into a suitable expression vector that is in turn used to transform a host cell to produce the polypeptide or RNA.

The term “having at least 80% identity” refers to an amino acid sequence or polynucleotide sequence that is at least 80% (e.g., at least 85%, at least 90%, at least 91%, at least 92%, at least 93%, at least 94%, at least 95%, at least 96%, at least 97%, at least 98%, or at least 99%) identical to the corresponding amino acid sequence or polynucleotide sequence. In some embodiments, the amino acid sequence or polynucleotide sequence having at least 80% identity is 100% identical to the corresponding amino acid sequence or polynucleotide sequence.

The amino acid sequence of SEQ ID NO: 1 corresponds to the amino acid sequence of EntD derived from E. coli MG1655. In some embodiments, the polypeptide has the amino acid sequence of SEQ ID NO: 1. In other embodiments, the polypeptide is a homologue of EntD having an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 1.

The terms “homolog,” “homologue,” and “homologous” as used herein refer to a polynucleotide or a polypeptide comprising a sequence that is at least about 80% homologous to the corresponding polynucleotide or polypeptide sequence. One of ordinary skill in the art is well aware of methods to determine homology between two or more sequences. Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a first sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of a second sequence. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm, such as BLAST (Altschul et al., J. Mol. Biol., 215(3): 403-410 (1990)). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3,4, 5, or 6 (Needleman and Wunsch, J. Mol. Biol., 48: 444-453 (1970)). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg, BMC Bioinformatics, 6: 278 (2005); Altschul et al., FEBS J., 272(20): 5101-5109 (2005)).

In the methods of the invention, the amino acid sequence having at least 80% identity to the amino acid sequence of SEQ ID NO: 1 encodes a polypeptide having PPTase activity. The term “phosphopanthetheinyl transferase” refers to a molecule, e.g., an enzyme, which catalyzes the transfer of a 4′-phosphopantetheine group from a donor compound to a substrate. Phosphopanthetheinyl transferases include natural enzymes, recombinant enzymes, synthetic enzymes, and active fragments thereof. The transfer of a 4′-phosphopantetheine group from a donor compound to a substrate is often referred to as “phosphopantetheinylating” a substrate.

The identity of the PPTase having at least 80% identity to the amino acid sequence of SEQ ID NO: 1 is not particularly limited, and one of ordinary skill in the art can readily identify homologues of EntD using the methods described herein as well as methods known in the art. In some embodiments, the PPTase having at least 80% identity to the amino acid sequence of EntD from E. coli MG1655 (i.e., SEQ ID NO: 1) is a PPTase as set forth in Table 1. Unless otherwise indicated, the accession numbers referenced herein are derived from the National Center for Biotechnology Information (NCBI) database maintained by the National Institute of Health, U.S.A.

TABLE 1

| | | GeneBank | Sequence | Amino Acid |
|---|---|---|---|---|
| Organism | Strain | Gene ID | Identifier | Identity¹ |
| E. coli | O157:H7 EDL933 | 957588 | SEQ ID NO: 3 | 99% |
| Shigella sonnei | Ss046 | 3667596 | SEQ ID NO: 4 | 99% |
| Shigella flexneri | 5 str. 8401 | 4210109 | SEQ ID NO: 5 | 99% |
| Shigella boydii | Sb227 | 3779189 | SEQ ID NO: 6 | 98% |
| Shigella boydii | CDC 3083-94 | 6273086 | SEQ ID NO: 7 | 97% |
| E. coli | IAI39 | 7153311 | SEQ ID NO: 8 | 94% |
| E. coli | 536 | 4191844 | SEQ ID NO: 9 | 93% |
| E. coli | UMN026 | 7156695 | SEQ ID NO: 10 | 92% |

¹determined using the BLAST program available on the NCBI website

The donor compound can be a natural or synthetic compound comprising a 4′-phosphopantetheine moiety. In preferred embodiments, the donor compound is coenzyme A (CoA).

A preferred substrate for PPTase is a polypeptide having carboxylic acid activity. Accordingly, in preferred embodiments of the invention, the method of producing a fatty aldehyde or a fatty alcohol in a host cell further includes expressing a polynucleotide encoding a polypeptide having carboxylic acid reductase activity, the identity of which is not particularly limited. Exemplary polypeptides having carboxylic acid reductase activity which are suitable for use in the methods of the present invention are disclosed, for example, in International Patent Application Publications WO 2010/062480 and WO 2010/042664. In some embodiments, the polypeptide having carboxylic acid reductase activity is CarA (SEQ ID NO: 11) or CarB (SEQ ID NO: 12) from M. smegmatis. In other embodiments, the polypeptide having carboxylic acid reductase activity is FadD9 from M. tuberculosis (SEQ ID NO: 13). In still other embodiments, the polypeptide having carboxylic acid reductase activity is CAR from Nocardia sp. NRRL 5646 (SEQ ID NO: 14). In yet other embodiments, the polypeptide having carboxylic acid reductase activity is a CAR from Mycobacterium sp. JLS (SEQ ID NO: 15) or Streptomyces griseus (SEQ ID NO: 16). The terms “carboxylic acid reductase,” “CAR,” and “fatty aldehyde biosynthetic polypeptide” are used interchangeably herein.

The invention also provides a method for transferring PPT to a substrate having carboxylic acid reductase activity. In one embodiment, the method comprises incubating a PPTase polypeptide comprising an amino acid sequence having at least 80% sequence identity to the amino acid sequence of SEQ ID NO: 1 with the substrate under conditions suitable for transfer of PPT, thereby transferring PPT to the substrate having carboxylic acid reductase activity.

In some embodiments, the polypeptide is a fragment of any of the polypeptides described herein. The term “fragment” refers to a shorter portion of a full-length polypeptide or protein ranging in size from four amino acid residues to the entire amino acid sequence minus one amino acid residue. In certain embodiments of the invention, a fragment refers to the entire amino acid sequence of a domain of a polypeptide or protein (e.g., a substrate binding domain or a catalytic domain).

In some embodiments, the polypeptide is a mutant or a variant of any of the polypeptides described herein. The terms “mutant” and “variant” as used herein refer to a polypeptide having an amino acid sequence that differs from a wild-type polypeptide by at least one amino acid. For example, the mutant can comprise one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions.

Preferred fragments or mutants of a polypeptide retain some or all of the biological function (e.g., enzymatic activity) of the corresponding wild-type polypeptide. In some embodiments, the fragment or mutant retains at least 75%, at least 80%, at least 90%, at least 95%, or at least 98% or more of the biological function of the corresponding wild-type polypeptide. In other embodiments, the fragment or mutant retains about 100% of the biological function of the corresponding wild-type polypeptide. Guidance in determining which amino acid residues may be substituted, inserted, or deleted without affecting biological activity may be found using computer programs well known in the art, for example, LASERGENE™ software (DNASTAR, Inc., Madison, Wis.).

In yet other embodiments, a fragment or mutant exhibits increased biological function as compared to a corresponding wild-type polypeptide. For example, a fragment or mutant may display at least a 10%, at least a 25%, at least a 50%, at least a 75%, or at least a 90% improvement in enzymatic activity as compared to the corresponding wild-type polypeptide. In other embodiments, the fragment or mutant displays at least 100% (e.g., at least 200%, or at least 500%) improvement in enzymatic activity as compared to the corresponding wild-type polypeptide.

It is understood that the polypeptides described herein may have additional conservative or non-essential amino acid substitutions, which do not have a substantial effect on the polypeptide function. Whether or not a particular substitution will be tolerated (i.e., will not adversely affect desired biological function, such as PPTase or carboxylic acid reductase activity) can be determined as described in Bowie et al. (Science, 247: 1306-1310 (1990)). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be prepared by using random and site-directed mutagenesis. Random and site-directed mutagenesis are described in, for example, Arnold, Curr. Opin. Biotech., 4: 450-455 (1993).

Random mutagenesis can be achieved using error prone PCR (see, e.g., Leung et al., Technique, 1: 11-15 (1989); and Caldwell et al., PCR Methods Applic., 2: 28-33 (1992)). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., a polynucleotide sequence encoding a PPTase) are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized, 30 pmole of each PCR primer, a reaction buffer comprising 50 mM KCl, 10 mM Tris HCl (pH 8.3), 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector, and the activities of the polypeptides encoded by the mutagenized nucleic acids are evaluated.

Site-directed mutagenesis can be achieved using oligonucleotide-directed mutagenesis to generate site-specific mutations in any cloned DNA of interest. Oligonucleotide mutagenesis is described in, for example, Reidhaar-Olson et al., Science, 241: 53-57 (1988). Briefly, in such procedures a plurality of double stranded oligonucleotides bearing one or more mutations to be introduced into the cloned DNA are synthesized and inserted into the cloned DNA to be mutagenized (e.g., a polynucleotide sequence encoding a PPTase). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequences in vitro as a result of random fragmentation of the DNA molecule based on sequence homology. This is followed by fixation of the crossover by primer extension in a PCR reaction. Sexual PCR mutagenesis is described in, for example, Stemmer, Proc. Natl. Acad. Sci., U.S.A., 91: 10747-10751 (1994).

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., a polynucleotide sequence encoding a PPTase) in one of these strains will eventually generate random mutations within the DNA. Mutator strains suitable for use for in vivo mutagenesis are described in, for example, International Patent Application Publication No. WO 1991/016427.

Variants can also be generated using cassette mutagenesis. In cassette mutagenesis, a small region of a double-stranded DNA molecule is replaced with a synthetic oligonucleotide “cassette” that differs from the native sequence. The oligonucleotide often contains a completely and/or partially randomized native sequence.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., Proc. Natl. Acad. Sci., U.S.A., 89: 7811-7815 (1992).

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res, 11: 1548-1552 (1993).

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

The invention also provides a recombinant host cell comprising (a) a polynucleotide sequence encoding a PPTase comprising an amino acid sequence having at least 80% identity to the amino acid sequence of SEQ ID NO: 1 and (b) a polynucleotide encoding a polypeptide having carboxylic acid reductase activity, wherein the recombinant host cell is capable of producing a fatty aldehyde or a fatty alcohol. In the embodiments wherein the polynucleotide sequence encodes an endogenous PPTase, the endogenous PPTase is overexpressed.

The invention further provides a recombinant host cell comprising (a) a polynucleotide sequence having at least 80% identity to the polynucleotide sequence of SEQ ID NO: 2 and (b) a polynucleotide encoding a polypeptide having carboxylic acid reductase activity, wherein the recombinant host cell is capable of producing a fatty aldehyde or a fatty alcohol. In the embodiments wherein the polynucleotide sequence encodes an endogenous PPTase, the endogenous PPTase is overexpressed.

As used herein, a “host cell” is a cell used to produce a product described herein (e.g., a fatty aldehyde or a fatty alcohol). In any of the aspects of the invention described herein, the host cell can be selected from the group consisting of a mammalian cell, plant cell, insect cell, fungus cell (e.g., a filamentous fungus cell or a yeast cell), and bacterial cell.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In other embodiments, the host cell is a Bacillus lentus cell, a Bacillus brevis cell, a Bacillus stearothermophilus cell, a Bacillus lichen formis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell.

In yet other embodiments, the host cell is an Actinomycetes cell.

In some embodiments, the host cell is a Saccharomyces cerevisiae cell. In some embodiments, the host cell is a Saccharomyces cerevisiae cell.

In still other embodiments, the host cell is a CHO cell, a COS cell, a VERO cell, a BHK cell, a HeLa cell, a Cv1 cell, an MDCK cell, a 293 cell, a 3T3 cell, or a PC12 cell.

In other embodiments, the host cell is a cell from an eukaryotic plant, algae, cyanobacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Avabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridiuthermocellum, Penicillium chrysogenum, Pichia pastoris, Saccharomyces cerevisiae, Schizosaccharomyces pombe, Pseudomonasjluorescens, or Zymomonas mobilis.

In certain preferred embodiments, the host cell is an E. coli cell. In some embodiments, the E. coli cell is a strain B, a strain C, a strain K, or a strain W E. coli cell.

In certain embodiments wherein the host cell is an E. coli host cell, the PPTase comprises an amino acid sequence other than the amino acid sequence of SEQ ID NO: 1, such as a homologue, fragment, or mutant of EntD.

In other embodiments wherein the host cell is an E. coli host cell and the polynucleotide sequence encodes an endogenous PPTase, the endogenous PPTase is overexpressed. An “endogenous PPTase” as used herein refers to a PPTase encoded by the genome of a wild-type host cell. For example, if the host cell is E. coli strain MG1655 and the polynucleotide sequence encodes the EntD PPTase consisting of the amino acid sequence of SEQ ID NO: 1, then the EntD PPTase is overexpressed.

In the embodiments of the invention wherein the polynucleotide sequence encodes an endogenous PPTase, the endogenous PPTase can be overexpressed by any suitable means. As used herein, “overexpress” means to express or cause to be expressed a polynucleotide, polypeptide, or hydrocarbon in a cell at a greater concentration than is normally expressed in a corresponding wild-type cell under the same conditions. For example, a polynucleotide can be “overexpressed” in a recombinant host cell when the polynucleotide is present in a greater concentration in the recombinant host cell as compared to its concentration in a non-recombinant host cell of the same species under the same conditions.

The term “increasing the level of expression of an endogenous PPTase” means to cause the overexpression of a polynucleotide sequence of an endogenous PPTase, or to cause the overexpression of an endogenous PPTase polypeptide sequence. The degree of overexpression can be about 1.5-fold or more, about 2-fold or more, about 3-fold or more, about 5-fold or more, about 10-fold or more, about 20-fold or more, about 50-fold or more, about 100-fold or more, or any range therein.

The term “increasing the level of activity of an endogenous PPTase” means to enhance the biochemical or biological function (e.g., enzymatic activity) of an endogenous PPTase. The degree of enhanced activity can be about 10% or more, about 20% or more, about 50% or more, about 75% or more, about 100% or more, about 200% or more, about 500% or more, about 1000% or more, or any range therein.

In some embodiments, overexpression of an endogenous PPTase is achieved by the use of an exogenous regulatory element. The term “exogenous regulatory element” generally refers to a regulatory element originating outside of the host cell. However, in certain embodiments, the term “exogenous regulatory element” can refer to a regulatory element derived from the host cell whose function is replicated or usurped for the purpose of controlling the expression of an endogenous PPTase. For example, if the host cell is an E. coli cell, and the PPTase is an endogenous PPTase, then expression of the endogenous PPTase can be controlled by a promoter derived from another E. coli gene.

In some embodiments, the exogenous regulatory element that causes an increase in the level of expression and/or activity of an endogenous PPTase is a chemical compound, such as a small molecule. As used herein, the term “small molecule” refers to a non-biological substance or compound having a molecular weight of less than about 1,000 g/mol.

In other embodiments, an increase in the level of expression and/or activity of an endogenous PPTase is effected by providing for the activation of another gene whose expression, in turn, regulates the expression and/or activity of an endogenous PPTase.

In some embodiments, the exogenous regulatory element which controls the expression of an endogenous polynucleotide encoding a PPTase is an expression control sequence which is operably linked to the endogenous polynucleotide by recombinant integration into the genome of the host cell. In certain embodiments, the expression control sequence is integrated into a host cell chromosome by homologous recombination using methods known in the art (e.g., Datsenko et al., Proc. Natl. Acad. Sci. U.S.A., 97(12): 6640-6645 (2000)).

Expression control sequences are known in the art and include, for example, promoters, enhancers, polyadenylation signals, transcription terminators, internal ribosome entry sites (IRES), and the like, that provide for the expression of the polynucleotide sequence in a host cell. Expression control sequences interact specifically with cellular proteins involved in transcription (Maniatis et al., Science, 236: 1237-1245 (1987)). Exemplary expression control sequences are described in, for example, Goeddel, Gene Expression Technology: Methods in Enzymology, Vol. 185, Academic Press, San Diego, Calif. (1990).

In the methods of the invention, an expression control sequence is operably linked to a polynucleotide sequence. By “operably linked” is meant that a polynucleotide sequence and an expression control sequence(s) are connected in such a way as to permit gene expression when the appropriate molecules (e.g., transcriptional activator proteins) are bound to the expression control sequence(s). Operably linked promoters are located upstream of the selected polynucleotide sequence in terms of the direction of transcription and translation. Operably linked enhancers can be located upstream, within, or downstream of the selected polynucleotide.

In some embodiments, the polynucleotide sequence is provided to the host cell by way of a recombinant vector, which comprises a promoter operably linked to the polynucleotide sequence. In certain embodiments, the promoter is a developmentally-regulated, an organelle-specific, a tissue-specific, an inducible, a constitutive, or a cell-specific promoter.

As used herein, the term “vector” refers to a nucleic acid molecule capable of transporting another nucleic acid, i.e., a polynucleotide sequence, to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids,” which refer generally to circular double stranded DNA loops that, in their vector form, are not bound to the chromosome. The terms “plasmid” and “vector” are used interchangeably herein, inasmuch as a plasmid is the most commonly used form of vector. However, also included are such other forms of expression vectors that serve equivalent functions and that become known in the art subsequently hereto.

In some embodiments, the recombinant vector comprises at least one sequence selected from the group consisting of (a) an expression control sequence operatively coupled to the polynucleotide sequence; (b) a selection marker operatively coupled to the polynucleotide sequence; (c) a marker sequence operatively coupled to the polynucleotide sequence; (d) a purification moiety operatively coupled to the polynucleotide sequence; (e) a secretion sequence operatively coupled to the polynucleotide sequence; and (f) a targeting sequence operatively coupled to the polynucleotide sequence.

The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech, Inc., Piscataway, N.J.; Smith et al., Gene, 67: 31-40 (1988)), pMAL (New England Biolabs, Beverly, Mass.), and pRITS (Pharmacia Biotech, Inc., Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al., Gene, 69: 301-315 (1988)) and PET 11d (Studier et al., Gene Expression Technology: Methods in Enzymology 185, Academic Press, San Diego, Calif., pp. 60-89 (1990)). Target gene expression from the pTrc vector relies on host RNA polymerase transcription from a hybrid trp-lac fusion promoter. Target gene expression from the PET 11d vector relies on transcription from a T7 gn10-lac fusion promoter mediated by a coexpressed viral RNA polymerase (T7 gn1). This viral polymerase is supplied by host strain BL21(DE3) or HMS174(DE3) from a resident λ prophage harboring a T7 gni gene under the transcriptional control of the lacUV 5 promoter.

In certain embodiments, a polynucleotide sequence of the invention is operably linked to a promoter derived from bacteriophage T5.

One strategy to maximize recombinant polypeptide expression is to express the polypeptide in a host cell with an impaired capacity to proteolytically cleave the recombinant polypeptide (see, e.g., Gottesman, Gene Expression Technology: Methods in Enzymology 185, Academic Press, San Diego, Calif., pp. 119-128 (1990)). Another strategy is to alter the nucleic acid sequence to be inserted into an expression vector so that the individual codons for each amino acid are those preferentially utilized in the host cell (Wada et al., Nucleic Acids Res., 20: 2111-2118 (1992)). Such alteration of nucleic acid sequences can be carried out by standard DNA synthesis techniques.

In certain embodiments, the host cell is a yeast cell, and the expression vector is a yeast expression vector. Examples of vectors for expression in yeast S. cerevisiae include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corp., San Diego, Calif.), and picZ (Invitrogen Corp., San Diego, Calif.).

In other embodiments, the host cell is an insect cell, and the expression vector is a baculovirus expression vector. Baculovirus vectors available for expression of proteins in cultured insect cells (e.g., Sf9 cells) include, for example, the pAc series (Smith et al., Mol. Cell Biol., 3: 2156-2165 (1983)) and the pVL series (Lucklow et al., Virology, 170: 31-39 (1989)).

In yet another embodiment, the polynucleotide sequences described herein can be expressed in mammalian cells using a mammalian expression vector. Examples of mammalian expression vectors include pCDM8 (Seed, Nature, 329: 840 (1987)) and pMT2PC (Kaufinan et al., EMBO J., 6: 187-195 (1987)). In some embodiments, expression of a polynucleotide sequence of the invention from a mammalian expression vector is controlled by viral regulatory elements, such as a promoter derived from polyoma, Adenovirus 2, cytomegalovirus, and Simian Virus 40. Other suitable expression systems for both prokaryotic and eukaryotic cells are well known in the art; see, e.g., Sambrook et al., “Molecular Cloning: A Laboratory Manual,” second edition, Cold Spring Harbor Laboratory, (1989).

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug.

Similarly, for stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to an antibiotic) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by growth in the presence of an appropriate selection drug.

As used herein, the term “conditions permissive for the production” means any conditions that allow a host cell to produce a desired product, such as a fatty aldehyde or a fatty alcohol. Similarly, the term “conditions in which the polynucleotide sequence of a vector is expressed” means any conditions that allow a host cell to synthesize a polypeptide. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source that can be metabolized by a host cell directly. In addition, enzymes can be used in the medium to facilitate the mobilization (e.g., the depolymerization of starch or cellulose to fermentable sugars) and subsequent metabolism of the carbon source.

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, and turanose; cellulosic material and variants such as methyl cellulose and sodium carboxymethyl cellulose; saturated or unsaturated fatty acid esters, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into a biofuel. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

In preferred embodiments of the invention, the host cell is cultured in a culture medium comprising at least one biological substrate for a polypeptide having CAR activity. In some embodiments, the medium comprises a fatty acid or a derivative thereof, such as a C₆-C₂₆ fatty acid. In certain embodiments, the fatty acid is a C₆, C₇, C₈, C₉, C₁₀, C₁₁, C₁₂, C₁₃, C₁₄, C₁₅, C₁₆, C₁₇, or C₁₈ fatty acid. In some embodiments, the medium comprises two or more (e.g., three or more, four or more, five or more) fatty acids or derivatives thereof, such as C₆-C₂₆ fatty acids. In certain embodiments, the medium comprises two or more (e.g., three or more, four or more, five or more) fatty acids selected from the group consisting of a C₆, C₇, C₈, C₉, C₁₀, C₁₁, C₁₂, C₁₃, C₁₄, C₁₅, C₁₆, C₁₇, and C₁₈ fatty acids. In any embodiment, the fatty acid substrate can be saturated or unsaturated.

To determine if conditions are sufficient to allow production of a product or expression of a polypeptide, a host cell can be cultured, for example, for about 4, 8, 12, 24, 36, 48, 72, or more hours. During and/or after culturing, samples can be obtained and analyzed to determine if the conditions allow production or expression. For example, the host cells in the sample or the medium in which the host cells were grown can be tested for the presence of a desired product. When testing for the presence of a fatty aldehyde or fatty alcohol, assays, such as, but not limited to, MS, thin layer chromatography (TLC), high-performance liquid chromatography (HPLC), liquid chromatography (LC), GC coupled with a flame ionization detector (FID), GC-MS, and LC-MS can be used. When testing for the expression of a polypeptide, techniques such as, but not limited to, Western blotting and dot blotting may be used.

The fatty aldehydes and fatty alcohols produced by the methods of invention generally are isolated from the host cell. The term “isolated” as used herein with respect to products, such as fatty aldehydes and fatty alcohols, refers to products that are separated from cellular components, cell culture media, or chemical or synthetic precursors. The fatty aldehydes and fatty alcohols produced by the methods described herein can be relatively immiscible in the fermentation broth, as well as in the cytoplasm. Therefore, the fatty aldehydes and fatty alcohols can collect in an organic phase either intracellularly or extracellularly. The collection of the products in the organic phase can lessen the impact of the fatty aldehyde or fatty alcohol on cellular function and can allow the host cell to produce more product.

In some embodiments, the fatty aldehydes and fatty alcohols produced by the methods of invention are purified. As used herein, the term “purify,” “purified,” or “purification” means the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of a fatty aldehyde or a fatty alcohol in a sample. For example, when a fatty aldehyde or a fatty alcohol is produced in a host cell, the fatty aldehyde or fatty alcohol can be purified by the removal of host cell proteins. After purification, the percentage of a fatty aldehyde or a fatty alcohol in the sample is increased.

As used herein, the terms “purify,” “purified,” and “purification” are relative terms which do not require absolute purity. Thus, for example, when a fatty aldehyde or a fatty alcohol is produced in host cells, a purified fatty aldehyde or a purified fatty alcohol is a fatty aldehyde or a fatty alcohol that is substantially separated from other cellular components (e.g., nucleic acids, polypeptides, lipids, carbohydrates, or other hydrocarbons). Additionally, a purified fatty aldehyde preparation or a purified fatty alcohol preparation is a fatty aldehyde preparation or a fatty alcohol preparation in which the fatty aldehyde or fatty alcohol is substantially free from contaminants, such as those that might be present following fermentation. In some embodiments, a fatty aldehyde or a fatty alcohol is purified when at least about 50% by weight of a sample is composed of the fatty aldehyde or the fatty alcohol. In other embodiments, a fatty aldehyde or a fatty alcohol is purified when at least about 60%, e.g., at least about 70%, at least about 80%, at least about 85%, at least about 90%, at least about 92% or more by weight of a sample is composed of the fatty aldehyde or the fatty alcohol. Alternatively, or in addition, a fatty aldehyde or a fatty alcohol is purified when less than about 100%, e.g., less than about 99%, less than about 98%, less than about 95%, less than about 90%, or less than about 80% by weight of a sample is composed of the fatty aldehyde or the fatty alcohol. Thus, a purified fatty aldehyde or a purified fatty alcohol can have a purity level bounded by any two of the above endpoints. For example, a fatty aldehyde or a fatty alcohol can be purified when at least about 80%-95%, at least about 85%-99%, or at least about 90%-98% of a sample is composed of the fatty aldehyde or the fatty alcohol.

In some embodiments, the fatty aldehyde or fatty alcohol is present in the extracellular environment, and the fatty aldehyde or fatty alcohol is isolated from the extracellular environment of the host cell. In certain embodiments, the fatty aldehyde or fatty alcohol is secreted from the host cell. In other embodiments, the fatty aldehyde or fatty alcohol is transported into the extracellular environment. In yet other embodiments, the fatty aldehyde or fatty alcohol is passively transported into the extracellular environment.

Fatty aldehydes and fatty alcohols can be isolated from a host cell using methods known in the art, such as those disclosed in International Patent Application Publications WO 2010/042664 and WO 2010/062480. One exemplary isolation process is a two phase (bi-phasic) separation process. This process involves fermenting the genetically engineered host cells under conditions sufficient to produce a fatty aldehyde or a fatty alcohol, allowing the fatty aldehyde or fatty alcohol to collect in an organic phase, and separating the organic phase from the aqueous fermentation broth. This method can be practiced in both batch and continuous fermentation processes.

Bi-phasic separation uses the relative immiscibility of fatty aldehydes and fatty alcohols to facilitate separation Immiscible refers to the relative inability of a compound to dissolve in water and is defined by the partition coefficient of a compound. As used herein, “partition coefficient” or “P,” is defined as the equilibrium concentration of a compound in an organic phase divided by the concentration at equilibrium in an aqueous phase (e.g., fermentation broth). In one embodiment of a bi-phasic system, the organic phase is formed by the fatty aldehyde or fatty alcohol during the production process. However, in certain embodiments, an organic phase can be provided, such as by providing a layer of octane, to facilitate product separation. When describing a two phase system, the partition characteristics of a compound can be described as logP. For example, a compound with a logP of 1 would partition 10:1 to the organic phase. A compound with a logP of −1 would partition 1:10 to the organic phase. One of ordinary skill in the art will appreciate that by choosing a fermentation broth and organic phase, such that the fatty aldehyde or fatty alcohol being produced has a high logP value, the fatty aldehyde or fatty alcohol can separate into the organic phase, even at very low concentrations, in the fermentation vessel.

The fatty aldehydes and fatty alcohols produced by the methods described herein can be relatively immiscible in the fermentation broth, as well as in the cytoplasm. Therefore, the fatty aldehyde and fatty alcohol can collect in an organic phase either intracellularly or extracellularly. The collection of the products in the organic phase can lessen the impact of the fatty aldehyde or fatty alcohol on cellular function and can allow the host cell to produce more product.

The methods described herein can result in the production of homogeneous compounds wherein at least about 60%, at least about 70%, at least about 80%, at least about 90%, or at least about 95%, of the fatty aldehydes or fatty alcohols produced will have carbon chain lengths that vary by less than 6 carbons, less than 5 carbons, less than 4 carbons, less than 3 carbons, or less than about 2 carbons. Alternatively, or in addition, the methods described herein can result in the production of homogeneous compounds wherein less than about 98%, less than about 95%, less than about 90%, less than about 80%, or less than about 70% of the fatty aldehydes or fatty alcohols produced will have carbon chain lengths that vary by less than 6 carbons, less than 5 carbons, less than 4 carbons, less than 3 carbons, or less than about 2 carbons. Thus, the fatty aldehydes and fatty alcohols can have a degree of homogeneity bounded by any two of the above endpoints. For example, the fatty aldehyde or fatty alcohol can have a degree of homogeneity wherein about 70%-95%, about 80%-98%, or about 90%-95% of the fatty aldehydes or fatty alcohols produced will have carbon chain lengths that vary by less than 6 carbons, less than 5 carbons, less than 4 carbons, less than 3 carbons, or less than about 2 carbons. These compounds can also be produced with a relatively uniform degree of saturation.

In some embodiments, the fatty aldehydes or fatty alcohols produced using methods described herein can contain between about 50% and about 90% carbon or between about 5% and about 25% hydrogen. In other embodiments, the fatty aldehydes or fatty alcohols produced using methods described herein can contain between about 65% and about 85% carbon or between about 10% and about 15% hydrogen.

In any aspect of the methods and compositions described herein, a fatty aldehyde or a fatty alcohol is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 g/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L, or a range bounded by any two of the foregoing values. In other embodiments, a fatty aldehyde or a fatty alcohol is produced at a titer of more than 2000 mg/L, more than 5000 mg/L, more than 10,000 mg/L, or higher.

In the methods of the invention, the production and isolation of fatty aldehydes and fatty alcohols can be enhanced by optimizing fermentation conditions.

EntD is known to transfer PPT to EntB and EntF, which are involved in producing the iron scavenging siderophore enterobactin (Gehring et al., Biochemistry, 36: 8495-8503 (1997)). EntD is only expressed under conditions of iron limitation, since the promoter for the fepA-entD operon contains binding sites for the ferric uptake regulator protein, Fur (Coderre et al., J. Gen. Microbiol., 135: 3043-3055 (1989)). Fur is a repressor of transcription of genes which contain a binding site for Fur (i.e., a “Fur box” or “iron box”) in their regulatory regions in the presence of its co-repressor, Fe²⁺. In the absence of Fe²⁺, Fur causes derepression of genes which contain a binding site for Fur (Andrews et al., FEMS Microbiol. Rev., 27: 215-237 (2003)).

High density growth is desirable in order to fulfill large scale commercial production of a chemical of interest in an engineered microorganism. Trace amounts of iron can support low density E. coli growth in shaker flasks, but higher amounts of iron are necessary for high density E. coli growth in a bioreactor. However, fatty aldehyde and fatty alcohol production in E. coli strains expressing a carboxylic acid reductase gene (e.g., CarB) and a thioesterase gene (e.g., ‘tesA) can be inhibited by the presence of iron (see, e.g., International Patent Application Publication WO 2010/062480).

In certain embodiments of the invention, the culture medium contains a low level of iron. The culture medium can contain less than about 500 μM iron, less than about 400 μM iron, less than about 300 μM iron, less than about 200 μM iron, less than about 150 μM iron, less than about 100 μM iron, less than about 90 μM iron, less than about 80 μM iron, less than about 70 μM iron, less than about 60 μM iron, or less than about 50 μM iron. Alternatively, or in addition, the culture medium can contain more than about 1 μM iron, more than about 5 μM iron, more than about 10 μM iron, more than about 20 μM iron, more than about 30 μM iron, or more than about 40 μM iron. Thus, the culture medium can have an iron content bounded by any two of the above endpoints. For example, the culture medium can have an iron content of about 5 μM to about 50 μM, about 10 μM to about 100 μM, about 100 μM to about 200 μM, or about 40 μM to about 400 μM. In certain embodiments, the medium does not contain iron.

In other embodiments, the culture medium contains a high level of iron. The culture medium can contain more than about 500 μM iron, more than about 1 mM iron, more than about 2 mM iron, more than about 5 mM iron, or more than about 10 mM iron. Alternatively, or in addition, the culture medium can contain less than about 25 mM iron, less than about 20 mM iron, or less than about 15 mM iron. Thus, the culture medium can have an iron content bounded by any two of the above endpoints. For example, the culture medium can have an iron content of about 500 μM to about 5 mM, about 2 mM to about 10 mM, or about 5 mM to about 20 mM.

In the methods of the invention, the production and isolation of fatty aldehydes and fatty alcohols can be enhanced by modifying the expression of one or more genes involved in iron metabolism. In some embodiments, the method further comprises modifying the expression of a gene encoding a polypeptide involved in iron metabolism. The identity of the gene is not particularly limited, and one of ordinary skill in the art is aware of candidate genes whose expression can be modified to facilitate growth in an iron-containing medium in order to enhance the production of fatty aldehydes and fatty alcohols. Exemplary polypeptides involved in iron metabolism suitable for use in the methods of the present invention are disclosed, for example, in Andrews et al. (supra). In certain embodiments, the gene encodes an iron uptake regulator. In particular embodiments, the gene is fur.

The invention also provides a method for relieving iron-induced inhibition of fatty aldehyde or fatty alcohol production in a host cell whose production of fatty aldehyde or fatty alcohol is sensitive to the amount of iron present in a medium for the host cell. The method comprises (a) expressing a polynucleotide sequence encoding a PPTase in the host cell and (b) culturing the host cell expressing the PPTase in a medium containing iron under conditions permissive for the production of a fatty aldehyde or a fatty alcohol. As a result of this method, expression of the PPTase causes an increase in the production of fatty aldehyde or fatty alcohol in the host cell as compared to the production of fatty aldehyde or fatty alcohol under the same conditions in the same host cell except for not expressing the PPTase. In certain embodiments, the PPTase comprises an amino acid sequence having at least 80% identity to the amino acid sequence of SEQ ID NO: 1. In other embodiments, the PPTase comprises an amino acid sequence having at least 80% identity to an amino acid sequence of SEQ ID NO: 17, 18, or 19.

The invention further provides a method for increasing the production of fatty aldehyde or fatty alcohol production in a host cell whose production of fatty aldehyde or fatty alcohol is sensitive to the amount of iron present in a medium for the host cell. The method comprises (a) expressing a polynucleotide sequence encoding a PPTase in the host cell, (b) culturing the host cell expressing the PPTase in a medium containing iron under conditions permissive for the production of a fatty aldehyde or a fatty alcohol, and (c) isolating the fatty aldehyde or fatty alcohol from the host cell. As a result of this method, expression of the PPTase results in an increase in the production of fatty aldehyde or fatty alcohol in the host cell as compared to the production of fatty aldehyde or fatty alcohol under the same conditions in the same host cell except for not expressing the PPTase. In certain embodiments, the PPTase comprises an amino acid sequence having at least 80% identity to the amino acid sequence of SEQ ID NO: 1. In other embodiments, the PPTase comprises an amino acid sequence having at least 80% identity to an amino acid sequence of SEQ ID NO: 17, 18, or 19.

Further provided is a method for relieving iron-induced inhibition of a polypeptide having carboxylic acid reductase activity in a host cell whose activity is sensitive to the amount of iron present in a medium for the host cell. The method comprises (a) expressing a polynucleotide sequence encoding a phosphopanthetheinyl transferase (PPTase) in the host cell, and (b) culturing the host cell expressing said PPTase in a medium containing iron. As a result of this method, the activity of a polypeptide having carboxylic acid reductase activity is increased upon expression of the PPTase as compared to the activity of the polypeptide having carboxylic acid reductase activity under the same conditions in the same host cell except for not expressing said PPTase. In certain embodiments, the PPTase comprises an amino acid sequence having at least 80% identity to the amino acid sequence of SEQ ID NO: 1. In other embodiments, the PPTase comprises an amino acid sequence having at least 80% identity to an amino acid sequence of SEQ ID NO: 17, 18, or 19.

In other embodiments, fermentation conditions are optimized to increase the percentage of the carbon source that is converted to hydrocarbon products. During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science 311: 1113 (2006); Venturi, FEMS Microbiol. Rev., 30: 274-291 (2006); and Reading et al., FEMS Microbiol. Lett., 254: 1-11 (2006)) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. Bacteriol., 182: 1127-1135 (2000)). UmuC is a DNA polymerase that can carry out translesion synthesis over non-coding lesions which commonly result from ultraviolet (UV) and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thereby minimizing the need for replication and maintenance pathways to be used while a fatty aldehyde or fatty alcohol is being made. Host cells can also be engineered to express umuC and umuD from E. coli in pBAD24 under the prpBCDE promoter system through de novo synthesis of this gene with the appropriate end-product production genes.

According to the methods of the invention, the efficiency by which an input carbon source is converted to product (e.g., fatty aldehyde or fatty alcohol) can be improved as compared to previously described processes. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other organic compounds and carbon sources. Typical efficiencies reported in the literature are approximately less than 5%. Host cells engineered to produce fatty aldehydes and fatty alcohols according to the methods of the invention can have an efficiency of at least about 1%, at least about 3%, at least about 5%, at least about 10%, at least about 15%, at least about 20%, at least about 25%, at least about 30%, or a range bounded by any two of the foregoing values. For example, the method of the invention results in an efficiency of about 5% to about 25%, about 10% to about 25%, about 10% to about 20%, about 15% to about 30%, or about 25% to about 30%. In other embodiments, the method of the invention results in greater than 30% efficiency.

The host cell can be additionally engineered to express a recombinant cellulosome, which can allow the host cell to use cellulosic material as a carbon source. Exemplary cellulosomes suitable for use in the methods of the invention include, e.g, the cellulosomes described in International Patent Application Publication WO 2008/100251. The host cell also can be engineered to assimilate carbon efficiently and use cellulosic materials as carbon sources according to methods described in U.S. Pat. Nos. 5,000,000; 5,028,539; 5,424,202; 5,482,846; and 5,602,030. In addition, the host cell can be engineered to express an invertase so that sucrose can be used as a carbon source.

In some embodiments of the fermentation methods of the invention, the fermentation chamber encloses a fermentation that is undergoing a continuous reduction, thereby creating a stable reductive environment. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty aldehydes and fatty alcohols.

For small scale production, the engineered host cells can be grown in batches of, for example, about 100 mL, 500 mL, 1 L, 2 L, 5 L, or 10 L; fermented; and induced to express a desired polynucleotide sequence, such as a polynucleotide sequence encoding a PPTase. For large scale production, the engineered host cells can be grown in batches of about 10 L, 100 L, 1000 L, 10,000 L, 100,000 L, 1,000,000 L or larger; fermented; and induced to express a desired polynucleotide sequence.

In some embodiments, a suitable production host, e.g., E. coli, harboring a plasmid containing the desired polynucleotide sequence encoding a PPTase and/or having an exogenous expression control sequence integrated into the E. coli chromosome and operably linked to a polynucleotide encoding an endogenouse PPTase can be incubated in a suitable reactor, for example a 1 L reactor, for 20 hours at 37° C. in M9 medium supplemented with 2% glucose, carbenicillin, and chloramphenicol. When the OD₆₀₀ of the culture reaches 0.9, the production host can be induced with IPTG. After incubation, the spent media can be extracted, and the organic phase can be examined for the presence of fatty aldehydes and fatty alcohols using, e.g., GC-MS.

In certain embodiments, after the first hour of induction, aliquots of no more than about 10% of the total cell volume can be removed each hour and allowed to sit without agitation to allow the fatty aldehydes and fatty alcohols to rise to the surface and undergo a spontaneous phase separation or precipitation. The fatty aldehydes and fatty alcohol components can then be collected, and the aqueous phase returned to the reaction chamber. The reaction chamber can be operated continuously. When the OD₆₀₀ drops below 0.6, the cells can be replaced with a new batch grown from a seed culture.

In the methods of the invention, the production and isolation of fatty aldehydes and fatty alcohols can be enhanced by modifying the expression of one or more genes involved in the regulation of fatty aldehyde and/or fatty alcohol production and secretion.

In some embodiments, the method further comprises modifying the expression of a gene encoding a fatty acid synthase in the host cell. As used herein, “fatty acid synthase” means any enzyme involved in fatty acid biosynthesis. In certain embodiments, modifying the expression of a gene encoding a fatty acid synthase includes expressing a gene encoding a fatty acid synthase in the host cell and/or increasing the expression or activity of an endogenous fatty acid synthase in the host cell. In alternate embodiments, modifying the expression of a gene encoding a fatty acid synthase includes attenuating a gene encoding a fatty acid synthase in the host cell and/or decreasing the expression or activity of an endogenous fatty acid synthase in the host cell. In some embodiments, the fatty acid synthase is a thioesterase. In particular embodiments, the thioesterase is encoded by tesA, tesA without leader sequence, tesB, fatB, fatB2, fatB3, fatA, or fatA1.

In certain embodiments, the method further comprises expressing a gene encoding a fatty aldehyde biosynthetic polypeptide in the host cell. Exemplary fatty aldehyde biosynthetic polypeptides suitable for use in the methods of the invention are disclosed, for example, in International Patent Application Publication WO 2010/042664. In preferred embodiments, the fatty aldehyde biosynthetic polypeptide has carboxylic acid reductase activity, e.g., fatty acid reductase activity.

In some embodiments, the method further comprises expressing a gene encoding a fatty alcohol biosynthetic polypeptide in the host cell. Exemplary fatty alcohol biosynthetic polypeptides suitable for use in the methods of the invention are disclosed, for example, in International Patent Application Publication WO 2010/062480. In certain embodiments, the fatty alcohol biosynthetic polypeptide is an alcohol dehydrogenase such as, but not limited to, AlrA of Acenitobacter sp. M-1 or AlrA homologs and endogenous E. coli alcohol dehydrogenases such as DkgA (NP_417485), DkgB (NP_414743), YjgB, (AAC77226), YdjL (AAC74846), YdjJ (NP_416288), AdhP (NP_415995), YhdH (NP_417719), YahK (NP_414859), YphC (AAC75598), and YqhD (446856).

As used herein, the term “alcohol dehydrogenase” is a peptide capable of catalyzing the conversion of a fatty aldehyde to an alcohol (e.g., fatty alcohol). One of ordinary skill in the art will appreciate that certain alcohol dehydrogenases are capable of catalyzing other reactions as well. For example, certain alcohol dehydrogenases will accept other substrates in addition to fatty aldehydes, and these non-specific alcohol dehydrogenases also are encompassed by the term “alcohol dehydrogenase.” Exemplary alcohol dehydrogenases suitable for use in the methods of the invention are disclosed, for example, in International Patent Application Publication WO 2010/062480.

In other embodiments, the host cell is genetically engineered to express an attenuated level of a fatty acid degradation enzyme relative to a wild-type host cell. As used herein, the term “fatty acid degradation enzyme” means an enzyme involved in the breakdown or conversion of a fatty acid or fatty acid derivative into another product, such as, but not limited to, an acyl-CoA synthase. In some embodiments, the host cell is genetically engineered to express an attenuated level of an acyl-CoA synthase relative to a wild-type host cell. In particular embodiments, the host cell expresses an attenuated level of an acyl-CoA synthase encoded by fadD, fadK, BH3103, yhfl, PJI-4354, EAV15023, fadD1, fadD2, RPC_4074, fadDD35, fadDD22, faa3p, or the gene encoding the protein ZP_0 1644857. In certain embodiments, the genetically engineered host cell comprises a knockout of one or more genes encoding a fatty acid degradation enzyme, such as the aforementioned acyl-CoA synthase genes.

In yet other embodiments, the method further comprises modifying the expression of a gene encoding a dehydratase/isomerase enzyme. In certain embodiments, modifying the expression of a gene encoding a dehydratase/isomerase enzyme includes expressing a gene encoding a dehydratase/isomerase enzyme in the host cell and/or increasing the expression or activity of an endogenous dehydratase/isomerase enzyme in the host cell. In other embodiments, a host cell is genetically engineered to express an attenuated level of a dehydratase/isomerase enzyme. In some embodiments, the host cell comprises a knockout of a dehydratase/isomerase enzyme. In certain embodiments, the gene encoding a dehydratase/isomerase enzyme is fabA.

In other embodiments, the method further comprises modifying the expression of a gene encoding a ketoacyl-ACP synthase. In certain embodiments, modifying the expression of a gene encoding a ketoacyl-ACP synthase includes expressing a gene encoding a ketoacyl-ACP synthase in the host cell and/or increasing the expression or activity of an endogenous ketoacyl-ACP synthase in the host cell. In other embodiments, a host cell is genetically engineered to express an attenuated level of a ketoacyl-ACP synthase. In certain embodiments, the host cell comprises a knockout of a ketoacyl-ACP synthase. In certain embodiments, the gene encoding a ketoacyl-ACP synthase is fabB. In yet other embodiments, the host cell is genetically engineered to express a modified level of a gene encoding a desaturase enzyme, such as desA.

In certain embodiments of the invention, the host cell is engineered to express (or overexpress) a transport protein. Transport proteins can export polypeptides and organic compounds (e.g., fatty aldehydes or fatty alcohols) out of a host cell. Many transport and efflux proteins serve to excrete a wide variety of compounds and can be modified to be selective for particular types of hydrocarbons. Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, and Rhodococcus erythropolis. Exemplary ABC transport proteins include, e.g., CER5, AtMRP5, AmiS2, and AtPGP1. In other embodiments, a host cell is chosen for its endogenous ability to secrete organic compounds. The efficiency of organic compound production and secretion into the host cell environment (e.g., culture medium, fermentation broth) can be expressed as a ratio of intracellular product to extracellular product. In some examples, the ratio can be about 5:1, 4:1, 3:1, 2:1, 1.1, 1.2, 1.3, 1.4, or 1.5.

The invention also provides a cell-free method for producing a fatty aldehyde. In one embodiment, a fatty aldehyde can be produced using a combination of purified polypeptides, such as a PPTase comprising an amino acid sequence having at least 80% identity to the amino acid sequence of SEQ ID NO: 1 and one or more fatty aldehyde biosynthetic polypeptides, and a substrate (e.g., a fatty acid). Exemplary fatty aldehyde biosynthetic polypeptides suitable for use in the cell-free methods of the invention are described, e.g., in International Patent Application Publication WO 2010/042664.

The invention also provides a cell-free method for producing a fatty alcohol. In one embodiment, a fatty alcohol can be produced using a combination of purified polypeptides, such as a PPTase comprising an amino acid sequence having at least 80% identity to the amino acid sequence of SEQ ID NO: 1 and one or more fatty alcohol biosynthetic polypeptides, and a substrate (e.g., a fatty acid or a fatty aldehyde). Exemplary fatty alcohol biosynthetic polypeptides suitable for use in the cell-free methods of the invention are described, e.g., in International Patent Application Publication WO 2010/062480. For example, a host cell can be engineered to express a PPTase and a fatty alcohol biosynthetic polypeptide as described herein. The host cell can be cultured under conditions suitable to allow expression of the polypeptides. Cell free extracts can then be generated using known methods. For example, the host cells can be lysed with detergents or by sonication. The expressed polypeptides can be purified using methods known in the art. After obtaining the cell free extracts, substrates described herein can be added to the cell free extracts and maintained under conditions to allow conversion of the substrates to fatty alcohols. The fatty alcohols can then be separated and purified using known techniques and the methods described herein.

The invention also provides a fatty aldehyde or a fatty alcohol produced by any of the methods described herein. A fatty aldehyde or a fatty alcohol produced by any of the methods described herein can be used directly as fuels, fuel additives, starting materials for production of other chemical compounds (e.g., polymers, surfactants, plastics, textiles, solvents, adhesives, etc.), or personal care additives. These compounds can also be used as feedstock for subsequent reactions, for example, hydrogenation, catalytic cracking (e.g., via hydrogenation, pyrolisis, or both), to make other products.

A used herein, the term “biofuel” refers to any fuel derived from biomass. Biofuels can be substituted for petroleum-based fuels. For example, biofuels are inclusive of transportation fuels (e.g., gasoline, diesel, jet fuel, etc.), heating fuels, and electricity-generating fuels. Biofuels are a renewable energy source. As used herein, the term “biodiesel” means a biofuel that can be a substitute of diesel, which is derived from petroleum. Biodiesel can be used in internal combustion diesel engines in either a pure form, which is referred to as “neat” biodiesel, or as a mixture in any concentration with petroleum-based diesel. Biodiesel can include esters or hydrocarbons, such as alcohols.

The invention also provides a surfactant or detergent comprising a fatty alcohol produced by any of the methods described herein. One of ordinary skill in the art will appreciate that, depending upon the intended purpose of the surfactant or detergent, different fatty alcohols can be produced and used. For example, when the fatty alcohols described herein are used as a feedstock for surfactant or detergent production, one of ordinary skill in the art will appreciate that the characteristics of the fatty alcohol feedstock will affect the characteristics of the surfactant or detergent produced. Hence, the characteristics of the surfactant or detergent product can be selected for by producing particular fatty alcohols for use as a feedstock.

A fatty alcohol-based surfactant and/or detergent described herein can be mixed with other surfactants and/or detergents well known in the art. In some embodiments, the mixture can include at least about 10%, at least about 15%, at least about 20%, at least about 30%, at least about 40%, at least about 50%, at least about 60%, or a range bounded by any two of the foregoing values, by weight of the fatty alcohol. In other examples, a surfactant or detergent composition can be made that includes at least about 5%, at least about 10%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of a fatty alcohol that includes a carbon chain that is 8, 9, 10, 11, 12, 13, 14, 15, 16, 17, 18, 19, 20, 21, or 22 carbons in length. Such surfactant or detergent compositions also can include at least one additive, such as a microemulsion or a surfactant or detergent from nonmicrobial sources such as plant oils or petroleum, which can be present in the amount of at least about 5%, at least about 10%, at least about 15%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of the fatty alcohol.

Fuel additives are used to enhance the performance of a fuel or engine. For example, fuel additives can be used to alter the freezing/gelling point, cloud point, lubricity, viscosity, oxidative stability, ignition quality, octane level, and/or flash point of a fuel. In the United States, all fuel additives must be registered with Environmental Protection Agency (EPA). The names of fuel additives and the companies that sell the fuel additives are publicly available by contacting the EPA or by viewing the EPA's website. One of ordinary skill in the art will appreciate that a fatty alcohol-based biofuel produced according to the methods described herein can be mixed with one or more fuel additives to impart a desired quality.

Bioproducts (e.g., fatty aldehydes, fatty alcohols, surfactants, and fuels) produced according to the methods of the invention can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588).

The ability to distinguish bioproducts from petroleum-based organic compounds is beneficial in tracking these materials in commerce. For example, organic compounds or chemicals comprising both biologically-based and petroleum-based carbon isotope profiles may be distinguished from organic compounds and chemicals made only of petroleum-based materials. Hence, the materials prepared in accordance with the inventive methods may be followed in commerce on the basis of their unique carbon isotope profile.

Bioproducts can be distinguished from petroleum-based organic compounds by comparing the stable carbon isotope ratio (¹³C/¹²C) in each fuel. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C₃ plants (the broadleaf), C₄ plants (the grasses), and marine carbonates all show significant differences in ¹³C/¹²C and the corresponding δ¹³C values. Furthermore, lipid matter of C₃ and C₄ plants analyze differently than materials derived from the carbohydrate components of the same plants as a consequence of the metabolic pathway.

The ¹³C measurement scale was originally defined by a zero set by Pee Dee Belemnite (PDB) limestone, where values are given in parts per thousand deviations from this material. The “δ¹³C” values are expressed in parts per thousand (per mil), abbreviated, % o, and are calculated as follows:

δ¹³C(% o)=[(¹³C/¹²C)ₛₐₘₚₗₑ−(¹³C/¹²C)_{standard}]/(¹³C/¹²C)_{standard}×1000

Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C₃”(or Calvin-Benson) photosynthetic cycle and those that incorporate the “C₄” (or Hatch-Slack) photosynthetic cycle.

In C₃ plants, the primary CO₂ fixation or carboxylation reaction involves the enzyme ribulose-1, 5-diphosphate carboxylase, and the first stable product is a 3-carbon compound. C₃ plants, such as hardwoods and conifers, are dominant in the temperate climate zones.

In C₄ plants, an additional carboxylation reaction involving another enzyme, phosphoenolpyruvate carboxylase, is the primary carboxylation reaction. The first stable carbon compound is a 4-carbon acid that is subsequently decarboxylated. The CO₂ thus released is refixed by the C₃ cycle. Examples of C₄ plants are tropical grasses, corn, and sugar cane.

Both C₄ and C₃ plants exhibit a range of ¹³C/¹²C isotopic ratios, but typical δ¹³C values for C₄ plants are about −7 to about −13, and typical δ¹³C values for C₃ plants are about −19 to about −27 (see, e.g., Stuiver et al., Radiocarbon, 19: 355 (1977)). Coal and petroleum fall generally in this latter range.

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46.

In some embodiments, a bioproduct produced according to the methods of the invention has a δ¹³C of about −30 or greater, about −28 or greater, about −27 or greater, about −20 or greater, about −18 or greater, about −15 or greater, about −13 or greater, or about −10 or greater. Alternatively, or in addition, a bioproduct has a δ¹³C of about −4 or less, about −5 or less, about −8 or less, about −10 or less, about −13 or less, about −15 or less, about −18 or less, or about −20 or less. Thus, the bioproduct can have a δ¹³C bounded by any two of the above endpoints. For example, the bioproduct can have a δ¹³C of about −30 to about −15, about −27 to about −19, about −25 to about −21, about −15 to about −5, about −13 to about −7, or about −13 to about −10. In some embodiments, the bioproduct can have a δ¹³C of about −10, −11, −12, or −12.3. In other embodiments, the bioproduct has a δ¹³C of about −15.4 or greater. In yet other embodiments, the bioproduct has a δ¹³C of about −15.4 to about −10.9, or a δ¹³C of about −13.92 to about −13.84.

Bioproducts can also be distinguished from petroleum-based organic compounds by comparing the amount of ¹⁴C in each compound. Because ¹⁴C has a nuclear half life of 5730 years, petroleum based fuels containing “older” carbon can be distinguished from bioproducts which contain “newer” carbon (see, e.g., Currie, “Source Apportionment of Atmospheric Particles”, Characterization of Environmental Particles, J. Buffle and H. P. van Leeuwen, Eds., Vol. I of the IUPAC Environmental Analytical Chemistry Series, Lewis Publishers, Inc., pp. 3-74 (1992)).

The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10⁻¹², with an approximate relaxation “half-life” of 7-10 years. This latter half-life must not be taken literally; rather, one must use the detailed atmospheric nuclear input/decay function to trace the variation of atmospheric and biospheric ¹⁴C since the onset of the nuclear age.

It is this latter biospheric ¹⁴C time characteristic that holds out the promise of annual dating of recent biospheric carbon. ¹⁴C can be measured by accelerator mass spectrometry (AMS), with results given in units of “fraction of modem carbon” (f_{M}). f_{M} is defined by National Institute of Standards and Technology (NIST) Standard Reference Materials (SRMs) 4990B and 4990C. As used herein, “fraction of modem carbon” or f_{M} has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

In some embodiments, a bioproduct produced according to the methods of the invention has a f_{M}¹⁴C of at least about 1, e.g., at least about 1.003, at least about 1.01, at least about 1.04, at least about 1.111, at least about 1.18, or at least about 1.124. Alternatively, or in addition, the bioproduct has an f_{M}¹⁴C of about 1.130 or less, e.g., about 1.124 or less, about 1.18 or less, about 1.111 or less, or about 1.04 or less. Thus, the bioproduct can have a f_{M}¹⁴C bounded by any two of the above endpoints. For example, the bioproduct can have a f_{M}¹⁴C of about 1.003 to about 1.124, a f_{M}¹⁴C of about 1.04 to about 1.18, or a f_{M}¹⁴C of about 1.111 to about 1.124.

Another measurement of ¹⁴C is known as the percent of modem carbon, i.e., pMC. For an archaeologist or geologist using ¹⁴C dates, AD 1950 equals “zero years old.” This also represents 100 pMC. “Bomb carbon” in the atmosphere reached almost twice the normal level in 1963 at the peak of thermo-nuclear weapons testing. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum-based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum-based products, the measured pMC value for that material will reflect the proportions of the two component types. For example, a material derived 100% from present day soybeans would have a radiocarbon signature near 107.5 pMC. If that material was diluted 50% with petroleum-based products, the resulting mixture would have a radiocarbon signature of approximately 54 pMC.

A biologically-based carbon content is derived by assigning “100%” equal to 107.5 pMC and “0%” equal to 0 pMC. For example, a sample measuring 99 pMC will provide an equivalent biologically-based carbon content of 93%. This value is referred to as the mean biologically-based carbon result and assumes that all of the components within the analyzed material originated either from present day biological material or petroleum-based material.

In some embodiments, a bioproduct produced according to the methods of the invention has a pMC of at least about 50, at least about 60, at least about 70, at least about 75, at least about 80, at least about 85, at least about 90, at least about 95, at least about 96, at least about 97, or at least about 98. Alternatively, or in addition, the bioproduct has a pMC of about 100 or less, about 99 or less, about 98 or less, about 96 or less, about 95 or less, about 90 or less, about 85 or less, or about 80 or less. Thus, the bioproduct can have a pMC bounded by any two of the above endpoints. For example, a bioproduct can have a pMC of about 50 to about 100; about 60 to about 100; about 70 to about 100; about 80 to about 100; about 85 to about 100; about 87 to about 98; or about 90 to about 95. In other embodiments, a bioproduct described herein has a pMC of about 90, about 91, about 92, about 93, about 94, or about 94.2.

The following examples further illustrate the invention but, of course, should not be construed as in any way limiting its scope.

### EXAMPLE 1

This example demonstrates enhanced fatty aldehyde and fatty alcohol production in the presence of high concentrations of iron.

The ferric uptake regulation (fur) gene encodes a global iron uptake regulator, and deletion of fur in E. coli results in lower concentrations of intracellular iron and iron-containing proteins (Abdul-Tehrani et al., J. Bacteriol., 181: 1415-1428 (1999)).

To determine the effect of fur deletion on fatty aldehyde and fatty alcohol production in E. coli, the fur gene of an E. coli DV2 strain was replaced with a kanamycin resistance gene amplified from pKD13 using primers furF (SEQ ID NO: 20) and furR (SEQ ID NO: 21), as described previously (e.g., Baba et al., Mol. Syst. Biol., 2: 2006.0008 (2006)). Gene replacement was verified by polymerase chain reaction (PCR) using primer furVF (SEQ ID NO: 22) and furVR (SEQ ID NO: 23). The fur mutant strain was designated “ALC2”. The primers used in this example are listed in Table 2.

TABLE 2

| | | Sequence |
|---|---|---|
| Primer | Sequence | Identifier |
| furF | GCAGGTTGGCTTTTCTCGTTCAGGCTGGC | SEQ ID NO: 20 |
| | TTATTTGCCTTCGTGCGCATGATTCCGGG | |
| | GATCCGTCGACC | |
| furR | CACTTCTTCTAATGAAGTGAACCGCTTAG | SEQ ID NO: 21 |
| | TAACAGGACAGATTCCGCATGTGTAGGCT | |
| | GGAGCTGCTTC | |
| furVF | ATTGAAGCCTGCCAGAGCGTGTTA | SEQ ID NO: 22 |
| furVR | CCTGATGTGATGCGGCGTAGACTC | SEQ ID NO: 23 |

Production of fatty aldehydes and fatty alcohols in E. coli can be facilitated by heterologous expression of a carboxylic acid reductase and a thioesterase. A plasmid (designated “p84.45BL”) was generated which contains carB from M. smegmatis and a ‘tesA Y145L mutant from E. coli downstream of a trc promoter in a pOP-80 vector. The pOP-80 vector has been described previously (International Patent Application Publication WO 2008/119082).

DV2 and ALC2 E. coli strains were transformed with p84.45BL and cultured at 37° C. in V9-B medium supplemented with spectinomycin (100 mg/L) in the presence or absence of 50 mg/L of iron (ferric ammonium citrate, CAS No. 1185-57-5). When the OD₆₀₀ reached ˜1.0, each culture was induced with 1 mM IPTG. At several time points post-induction, a sample of each culture was removed and extracted with butyl acetate. Fatty alcohol, fatty aldehyde, and fatty acid contents in the crude extracts were measured with GC-MS as described in International Patent Application Publication WO 2008/119082.

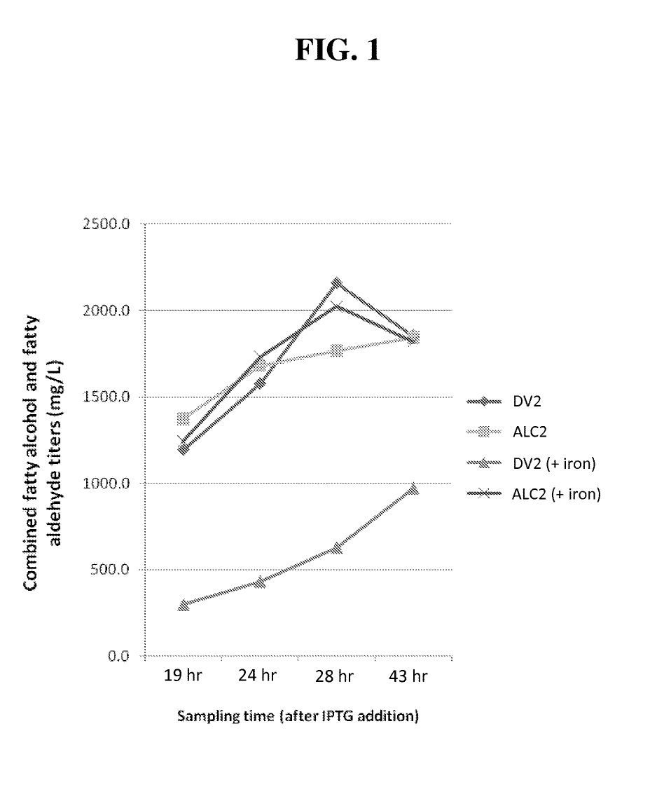

The fur mutant ALC2/p84.45BL strain produced much higher quantities of fatty aldehydes and fatty alcohols than the control DV2/p84.45BL strain when iron was present in the fermentation medium (FIG. 1). The levels of fatty aldehydes and fatty alcohols produced from the ALC2/p84.45BE strain in the presence of iron were comparable to the levels of fatty aldehydes and fatty alcohols produced by the DV2/p84.45BE strain in the absence of iron (FIG. 1). The levels of fatty aldehydes and fatty alcohols produced from the ALC2/p84.45BE strain did not appear to be affected by the presence of iron in fermentation medium (FIG. 1).

Qualitative differences in fatty alcohol, fatty aldehyde, and fatty acid production also were observed between the ALC2/p84.45BL and DV2/p84.45BL strains. In the presence of iron, the DV2/p84.45BL strain produced primarily C₈, C₁₀, and C₁₂ alcohols, but did not appear to produce C₁₄ and C₁₆ alcohols. In addition, large amounts of C₁₄ and C₁₆ fatty acids were produced from the DV2/p84.45BL strain, while no significant amounts of fatty acids were produced from the ALC2/p84.45BL strains.

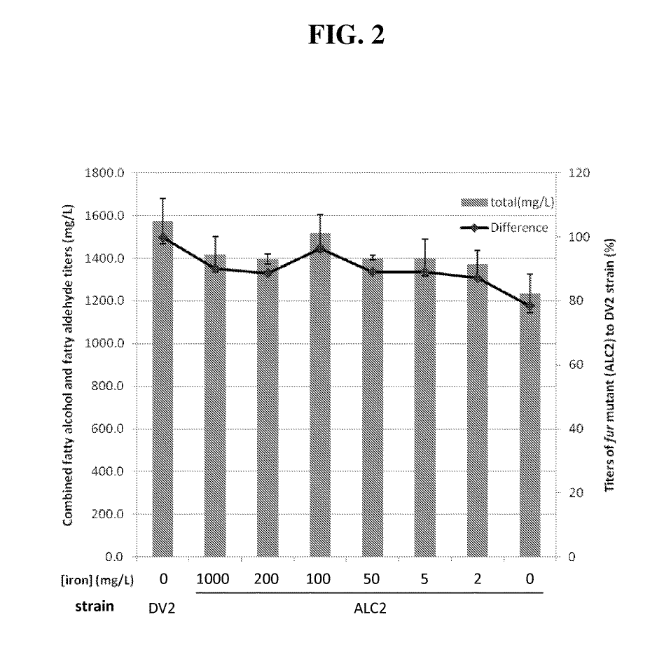

To test whether fatty aldehyde and fatty alcohol production in the fur mutant strain was affected by the concentration of iron, ALC2/p84.45BL transformants were cultured in the presence of several different concentrations of ferric ammonium citrate. After induction with IPTG, fatty aldehyde and fatty alcohol levels in the cultures were determined by GC-MS as described above. The levels of fatty aldehydes and fatty alcohols produced from ALC2/p84.45BL were slightly higher in medium containing iron as compared to medium lacking iron, although varying the concentration of iron from 2 mg/L to 1000 mg/L did not substantially affect production levels (FIG. 2).

The results of this example demonstrate that deletion of the fur gene facilitates fatty aldehyde and fatty alcohol production in E. coli in media containing high concentrations of iron.

### EXAMPLE 2

This example demonstrates that expression of the E. coli EntD phosphopantetheinyl transferase (PPTase) or a PPTase homologue can relieve the inhibition of fatty alcohol production induced by iron.

The results from Example 1 demonstrated that the presence of iron in the fermentation medium inhibits the production of fatty alcohols and fatty aldehydes in E. coli strains expressing CarB. Although excluding iron is a viable option for small scale fermentations (˜100 mL), its presence is essential for high density growth in large fermentations (e.g., in a bioreactor).

To determine the effect of EntD on fatty aldehyde and fatty alcohol production in an iron-containing medium, an E. coli strain in which entD is overexpressed was generated by cloning the entD gene between the EcoRI and HindIII sites of plasmid pBAD24 (Cronan, Plasmid, 55(2): 152-157 (2006)) using the EntD-for (SEQ ID NO: 24) and EntD-rev (SEQ ID NO: 25) primer set listed in Table 3. This plasmid, designated “pDG104,” contained the entD gene under the control of an inducible arabinose promoter.

TABLE 3

| | | Sequence |
|---|---|---|
| Primer | Sequence | Identifier |
| EntD-for | CAGGAGGAATTCACCATGGTCGATATG | SEQ ID NO: 24 |
| | AAAACTACGCATACCTCC | |
| EntD-rev | AGATGTAAGCTTTTAATCGTGTTGGCA | SEQ ID NO: 25 |
| | CAGCGTTATGACTAT | |

A DV2 E. coli strain was transformed with pDG104 or pBAD24 (empty vector). Transformants were grown in 2 mL of Luria-Bertani (LB) medium supplemented with spectinomycin (100 mg/L) and carbenicillin (100 mg/L) at 37° C. After overnight growth, 100 μL of culture was transferred into 2 mL of fresh LB supplemented with antibiotics. After 2-3 hours growth, 2 mL of culture was transferred into a 125 mL-flask containing 20 mL of M9 medium with 2% glucose supplemented with antibiotics, 1 μg/L thiamine, and 20 μL of the trace mineral solution described in Table 4.

TABLE 4
Trace mineral solution
(filter sterilized)

27 g/L FeCl₃•6H₂O
2 g/L ZnCl•4H₂O
2 g/L CaCl₂•6H₂O
2 g/L Na₂MoO₄•2H₂O
1.9 g/L CuSO₄•5H₂O
0.5 g/L H₃BO₃
100 mL/L concentrated HCl
q.s. Milli-Q water

When the OD₆₀₀ of the culture reached 1.0, 1 mM of IPTG and 10 mM of arabinose were added to each flask. After 20 hours of growth at 37° C., a 200 μL sample from each flask was removed, and fatty alcohols and fatty aldehydes were extracted with 400 μL butyl acetate. The crude extracts were analyzed directly with GC-MS as described in Example 1.

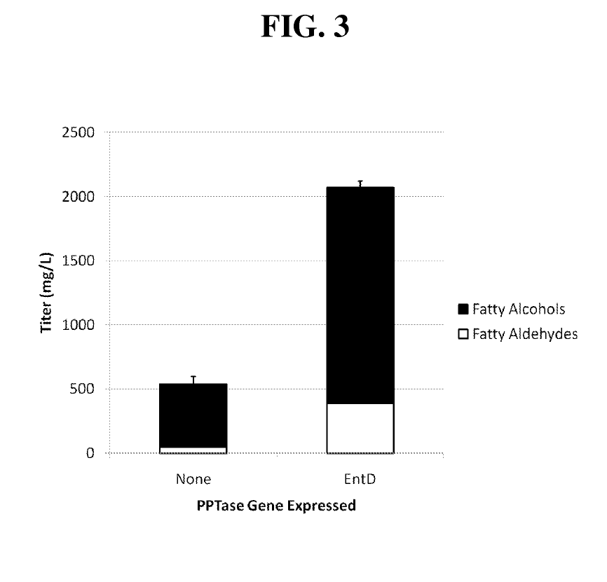

DV2 transformed with the control pBAD24 plasmid produced 500 mg/L or less total fatty alcohols and fatty aldehydes in the presence of iron (FIG. 3), which titer was similar to that of untransformed DV2. Inclusion of arabinose in the culture medium had no effect on titer produced by control transformants. In contrast, a DV2 strain transformed with pDG104 produced greater than 2000 mg/L total fatty alcohols and fatty aldehydes in the presence of iron during the first 20 hours of fermentation (FIG. 3). Titers were 10-20% lower if the arabinose inducer was omitted, thereby suggesting that low, background expression of EntD may be sufficient to activate a fraction of the CarB enzyme pool.

The results of this example demonstrate that overexpression of EntD relieves iron-induced inhibition of fatty alcohols and fatty aldehydes production in E. coli.

### EXAMPLE 3

This example demonstrates the construction of E. coli strains expressing various PPTases from diverse organisms.

Four E. coli strains were constructed in which various PPTases from diverse organisms were expressed from the E. coli chromosome at the same locus under the control of a T5 phage promoter. The PPTases selected for expression in E. coli in this example are listed in Table 5. The selected PPTases were from diverse bacterial clades, represented both gram negative and gram positive bacteria, and displayed a varying degree of amino acid identity as compared to EntD from E. coli MG1655.

TABLE 5

| | | | Amino | Amino | |
|---|---|---|---|---|---|
| | | | acid | acid | |
| PPTase | Organism | Gene | sequence | identity | Source |
| EntD | Escherichia coli | entD | SEQ ID | 100% | genomic DNA |
| | MG1655 | | NO: 1 | | |
| Sfp | Bacillus subtilis | sfp | SEQ ID | 23% | pMA_1001546 (SEQ |
| | ATCC 21332 | | NO: 17 | | ID NO: 26) |
| Ppt_{MC155} | Mycobacterium | MSME | SEQ ID | 35% | pDF14 (SEQ ID NO: |
| | smegmatis MC155 | G_2648 | NO: 18 | | 27) |
| PcpS | Pseudomonas | pcpS | SEQ ID | 51% | pJ204_38022 (SEQ ID |
| | aeruginosa | | NO: 19 | | NO: 28) |

To construct a promoter cassette to be integrated upstream of the endogenous entD gene of E. coli, a chloramphenicol resistance gene (cat)-T5 promoter cassette was amplified by PCR from a pKD3 plasmid template using primers cat-for (SEQ ID NO: 29) and cat-rev (SEQ ID NO: 30). The cat-rev primer contains the sequence for a promoter from phage T5. The primers used in this example are listed in Table 6.

TABLE 6

| | | Sequence |
|---|---|---|
| Primer | Sequence | Identifier |
| cat-for | AGCCGGGACGTACGTGGTATATGAGCGTAAACACCCACTTCTGA | SEQ ID NO: 29 |
| | TGCTAAGTGTAGGCTGGAGCTGCTTCG | |
| cat-rev | ATTCGAGACTGATGACAAACGCAAAACTGCCTGATGCGCTACGC | SEQ ID NO: 30 |
| | TTATCATTGAATCTATTATACAGAAAAATTTTCCTGAAAGCAAA | |
| | TAAATTTTTTATGATTGACATGGGAATTAGCCATGGTCC | |
| sfp-for | TGATAAGCGTAGCGCATCAGGCAGTTTTGCGTTTGTCATCAGTC | SEQ ID NO: 31 |
| | TCGAATATGAAGATTTACGGAATTTATATGGACCGCCCGCTTTC | |
| sfp-rev | AGGCACCTGCTTTACACTTTCGCCCG | SEQ ID NO: 32 |
| ppt_{MC155}-for | GCATCAGGCAGTTTTGCGTTTGTCATCAGTCTCGAATATGGGCA | SEQ ID NO: 33 |
| | CCGATAGCCTGTTGAGC | |
| ppt_{MC155}-rev | TCGCCCGTGGTCAGTGATGGCTGCGGGCGAATCGTACCAGATGT | SEQ ID NO: 34 |
| | TGTCAATTACAGGACAATCGCGGTCACC | |
| pcpS-for | TGATAAGCGTAGCGCATCAGGCAGTTTTGCGTTTGTCATCAGTC | SEQ ID NO: 35 |
| | TCGAATATGCGCGCGATGAACGACAGACTGC | |
| pcpS-rev | AGGCACCTGCTTTACACTTTCGCCCG | SEQ ID NO: 36 |
| sfpSOE-for | AGCCGGGACGTACGTGGTATATGAGCG | SEQ ID NO: 37 |
| sfpSOE-rev | AGGCACCTGCTTTACACTTTCGCCCG | SEQ ID NO: 38 |
| ppt_{MC155}SOE-for | AGCCGGGACGTACGTGGTATATGAGCG | SEQ ID NO: 39 |
| ppt_{MC155}SOE-rev | TCGCCCGTGGTCAGTGATGGCTG | SEQ ID NO: 40 |
| pcpSSOE-for | AGCCGGGACGTACGTGGTATATGAGCG | SEQ ID NO: 41 |
| pcpSSOE-rev | AGGCACCTGCTTTACACTTTCGCCCG | SEQ ID NO: 42 |
| ΔentD::cat-for | TGATAAGCGTAGCGCATCAGGCAGTTTTGCGTTTGTCATCAGTC | SEQ ID NO: 43 |
| | TCGAATGTGTAGGCTGGAGCTGCTTCG | |
| ΔentD::cat-rev | TCGCCCGTGGTCAGTGATGGCTGCGGGCGAATCGTACCAGATGT | SEQ ID NO: 44 |
| | TGTCAAGACATGGGAATTAGCCATGGTCC | |
| screening-for | GGCAAGCAGCAGCCGAAGAAGTA | SEQ ID NO: 45 |
| screening-rev | GGTGGCCATTCGTGGGACAGTATCC | SEQ ID NO: 46 |

To construct expression cassettes for sfp, pptMC155, and pcpS, each PPTase was PCR amplified from its respective source DNA listed in Table 5, using the corresponding gene-specific primer pairs listed in Table 6. Subsequently, each of the three PCR-amplified PPTase genes was individually spliced to the cat-T5 promoter cassette with splicing by overlapping extension (SOE)-PCR (see, e.g., Horton et al., Gene, 77: 61-68 (1989)) using the corresponding gene-specific SOE primer pairs listed in Table 6.

E. coli strains containing either the cat-T5 promoter cassette integrated upstream of the endogenous entD gene or the cat-T5 promoter expression cassette for sfp, pptMC155, or pcpS were generated as described previously (Datsenko et al., Proc. Natl. Acad. Sci. U.S.A., 97(12): 6640-6645 (2000)).

Briefly, a recipient E. coli V261 strain (MG1655 ΔfadE::FRT ΔfhuA::FRT ΔfabB::fabB[A329V]) was made electrocompetent and then transformed with 0.5 μL of helper plasmid pKD46. The cells were recovered in LB media without antibiotics at 32° C. for one hour, plated onto LB agar containing 100 μg/mL carbenicillin, and incubated at 32° C. overnight.

A colony of the recipient strain was then cultured at 32° C. in LB medium containing 100 μg/mL carbenicillin and 10 mM L-arabinose until the cells reached an OD₆₀₀ of 0.4-1.0, at which point the cells were transformed with 2-5 μL of a linear DNA cassette comprising the cat-T5 promoter cassette (for EntD expression) or the cat-T5 promoter cassette linked to sfp, pptMC155, or pcpS. The cells were recovered in LB media without antibiotics at 32° C. or 37° C. for one hour, plated onto LB agar containing chloramphenicol, and incubated at 32° C. or 37° C. overnight.

Individual colonies were screened to verify the presence of the correct integration cassette by colony PCR using the screening-for (SEQ ID NO: 45) and screening-rev (SEQ ID NO: 46) primer set.

Next, the cells were cured of the pKD46 helper plasmid by culturing for at least 3 hours at 42° C. in LB medium with no antibiotics and then streaking onto LB agar plates to isolate single colonies. Loss of the pKD46 plasmid was verified by streaking single colonies on LB plates containing 100 μg/mL carbenicillin at 32° C.

To remove the FRT-flanked antibiotic marker, cells were made electrocompetent and transformed with 0.5 μL pCP20 helper plasmid. The cells were recovered in LB medium with no antibiotics at 32° C. and then selected for the presence of pCP20 by plating onto LB agar supplemented with 100 μg/mL carbenicillin or 34 μg/mL chloramphenicol and incubating at 32° C.

Next, single colonies were selected, cultured at 42° C. for several hours in LB medium with no antibiotics, and then streaked on LB agar plates to isolate single colonies. Simultaneous loss of the FRT-flanked resistance gene and the pCP20 helper plasmid was verified by streaking single colonies on two plates, one which contained LB agar with 100 μg/mL carbenicillin or 34 μg/mL chloramphenicol to test for pCP20 loss, and another which contained LB agar with the appropriate antibiotic to test for chromosomal antibiotic resistance loss.

All strains were confirmed to contain the appropriate PPTase via colony PCR screening and sequencing using the screening-for (SEQ ID NO: 45) and screening-rev (SEQ ID NO: 46) primer set.

The results of this example demonstrate construction of E. coli strains expressing various PPTases from diverse organisms.

### EXAMPLE 4

This example demonstrates that PPTases from diverse organisms can enhance fatty alcohol production in an engineered microorganism.

Each of the four PPTase-expressing E. coli strains described in Example 3 were transformed with a plasmid designated “p7P36” (SEQ ID NO: 47) which facilitates fatty alcohol production. The p7P36 plasmid is based upon the pCL1920 plasmid and contains carB from M. smegmatis, 13G04 (an E. coli ‘tesA variant), and alrAadp1 (aldehyde reductase) from Acinetobacter sp. M1.

Three colonies from each PPTase-expressing strain were assessed for fatty alcohol production using the method described in Example 2, except that carbenicillin was not added to the growth medium, and arabinose was not added during the induction period.

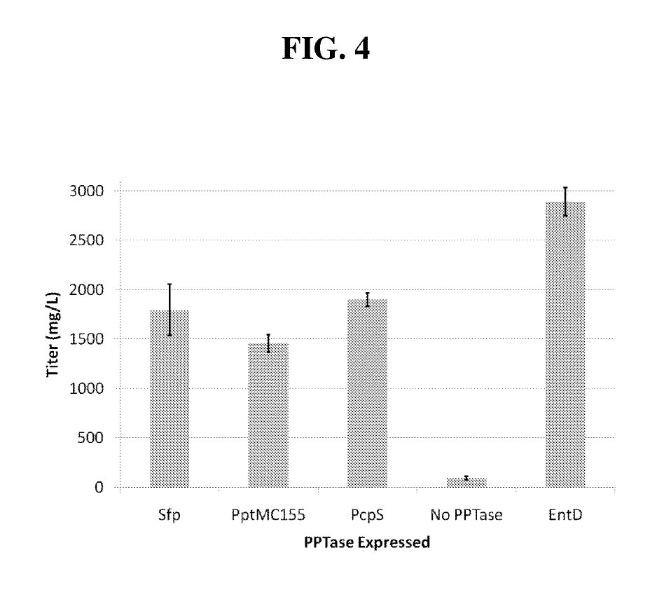

In the absence of exogenous PPTase, very little fatty alcohol production was observed (FIG. 4). In contrast, expression of EntD, Sfp, Ppt_{MC155}, or PcpS from the E. coli chromosome under the control of a phage T5 promoter led to substantial levels of fatty alcohol production (FIG. 4). Under the experimental conditions tested, expression of EntD led to the highest fatty alcohol production titers (˜2900 mg/L), followed by PcpS (˜1900 mg/L), Sfp (˜1800 mg/L), and then Ppt_{MC155} (4500 mg/L).

This results of this example demonstrate that PPTases from diverse organisms can enhance fatty alcohol production in E. coli, and that particularly high titers of fatty alcohols can be achieved by expression of EntD.

### EXAMPLE 5

This example demonstrates that PPTase activity is required to activate CarB.

To test the effect of entD on CarB activity, an in vitro enzyme assay was performed with CarB isolated from two E. coli strains. The first strain expressed EntD from the E. coli chromosome under the control of a phage T5 promoter (described in Examples 3 and 4) (hereinafter “+EntD”), and the second strain contained a deletion of the entD gene (hereinafter “−EntD”).

To construct the entD deletion cassette, plasmid pKD3 was used as a template for PCR using the ΔentD::cat-for (SEQ ID NO: 43) and ΔentD::cat-rev (SEQ ID NO: 44) primer pair listed in Table 6. The PCR product was then used to replace entD from E. coli strain V261 (MG1655 ΔfadE::FRT ΔfhuA::FRT ΔfabB::fabB[A329V]) with a chloramphenicol resistance cassette using the method described in Example 3 (Datsenko et al., supra).

N-terminal histidine-tagged CarB was expressed from a pCL1920 vector in +EntD and −EntD cells to generate CarB+EntD cells and CarB-EntD cells, respectively. The cultures were grown at 37° C. in FA-2 (minimum) medium supplemented with 100 μg/mL spectinomycin by a three-stage fermentation protocol. The cultures were grown to an OD₆₀₀ of approximately 1.6, induced with 1 mM IPTG, and incubated for additional 23 hours at 37° C.

To purify CarB, the cells were harvested by centrifugation and suspended in BUGBUSTER™ MasterMix (Novagen) lysis buffer containing a protease inhibitor cocktail solution. The cells were disrupted by French pressing, and the resulting homogenate was centrifuged to remove cellular debris. CarB in the resulting supernatant was purified with nickel-nitrilotriacetic acid (Ni-NTA) resin and either analyzed by SDS-PAGE or dialyzed against 20% (v/v) glycerol in 50 mM sodium phosphate buffer, pH 7.5, flash-frozen, and stored at −80° C.

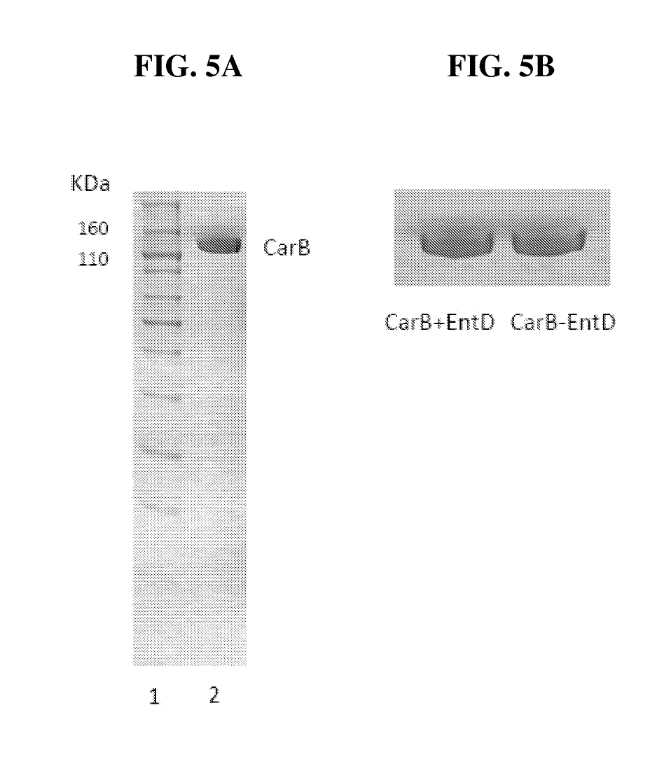

CarB purified from CarB+EntD cells displayed a high level of purity as assessed by SDS-PAGE and Coomassie blue staining (FIG. 5A). No apparent differences were observed between CarB purified from CarB+EntD cells as compared to CarB purified from CarB-EntD cells by SDS-PAGE and Coomassie blue staining (FIG. 5B).

The enzymatic activity of CarB purified from CarB+EntD and CarB-EntD strains was measured in 200 μL of a reaction mixture containing 5 mM benzoate, 0.2 mM NADPH, 1 mM ATP, 10 mM MgCl₂, 1 mM DTT, and CarB in 50 mM Tris buffer (pH 7.5). CarB activity was measured spectrophotometrically by following the decrease of NADPH absorbance at 340 nm at 25° C.

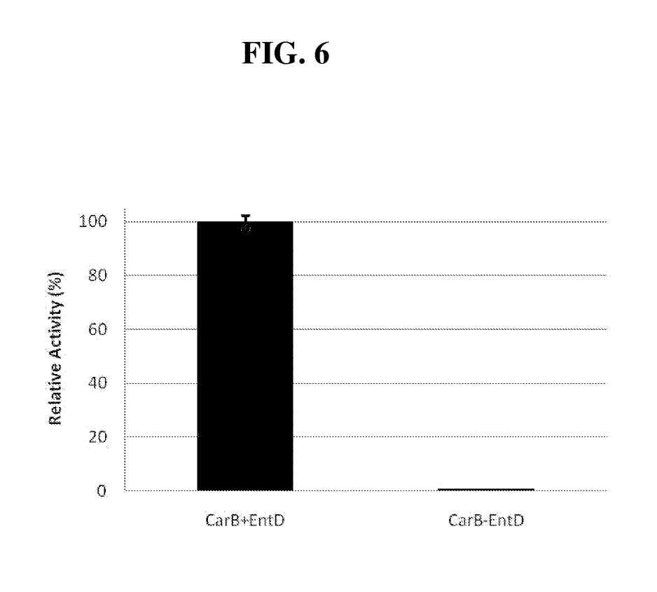

CarB purified from E. coli in which entD was deleted displayed only about 1.0% of CAR activity as compared to the CAR activity of CarB purified from E. coli overexpressing entD from a T5 promoter (FIG. 6).

To determine whether CarB purified from cells lacking entD could be activated, recombinant CarB purified from CarB-EntD cells as described above was incubated with 4-12 μM Sfp, 12 μM Coenzyme A, and 10 mM MgCl₂ in 50 mM Tris buffer (pH 7.5) at 37° C. After a 1 hour incubation, CarB was assayed for CAR activity as described above.

Incubation of CarB from the entD deletion strain with recombinant Sfp led to a full recovery of CarB activity, suggesting that Sfp can compensate for the absence of EntD in the activation of CarB.

The results of this example reflect a requirement for PPTase activity to activate CarB in E. coli.

### EXAMPLE 6

This example demonstrates a technique for enhanced production of fatty aldehydes and fatty alcohols in S. cerevisiae based upon a method described in U.S. Patent Application Publication 2010/0298612.

In order to provide for the expression of EntD and CarB in S. cerevisiae, an entD gene (e.g., SEQ ID NO: 2) is amplified by PCR and then cloned into the vector pESC-LEU (Stratagene, La Jolla, Calif.) downstream of the GAL10 promoter using the NotI and SpeI restriction sites, thereby generating a vector termed “pENTD.” A gene encoding a CarB polypeptide (e.g., SEQ ID NO: 12) is then amplified by PCR and cloned into pENTD downstream of the GAL1 promoter using the BamHI and SalI restriction sites, thereby generating a vector termed “pENTD_CARB.” The pENTD_CARB vector contains a 2 micron yeast origin and a LEU2 gene for selection in S. cerevisiae YPH499 (Stratagene, La Jolla, Calif.).

To determine the in vivo activity of CarB in recombinant S. cerevisiae host cells, recombinant S. cerevisiae strains comprising pENTD_CARB are inoculated in 5 mL of Yeast Nitrogen Base (YNB)-Leu containing 2% glucose (SD media) and grown at 30° C., overnight, until an OD₆₀₀ of approximately 3 is reached. Approximately 2.5 mL are then subcultured into 50 mL of SD media (i.e., 20× dilution to an OD of approximately 0.15) and grown at 30° C. for 8 hours until an OD₆₀₀ of approximately 1 is reached. Cell cultures are then centrifuged at approximately 3000-4000 RPM (e.g., using a F15B-8×50C rotor) for 10 minutes, and the supernatant is discarded. Residual medium is removed with a pipette, or the cells are washed with SG medium (YNB-Leu containing 2% galactose). The cell pellets are resuspended in 250 mL SG media (i.e., 5× dilution to achieve a starting culture having an OD₆₀₀ of approximately 0.2), and grown overnight at 30° C.

For extraction and identification of intracellular fatty aldehydes and fatty alcohols, 30-50 OD₆₀₀ units of cells are centrifuged, and the cell pellets are washed with 20 mL of 50 mM Tris-HCl pH 7.5. Cells are resuspended in 0.5 mL of 6.7% Na₂SO₄, and transferred into 2-mL tubes. 0.4 mL of isopropanol and 0.6 mL of hexane are added, and the mixture is vortexed for approximately 30 minutes, and then centrifuged for 2 minutes at 14,000 RPM using a bench top centrifuge (e.g., Eppendorf F45-25-11). The upper organic phase is collected and evaporated under a nitrogen stream. The remaining residue is derivatized with 100 μL Bis(Trimethylsilyl)-Trifluoroacetamide (BSTFA) at 37-60° C. for 1 hour, held at room temperature for another 3 to 12 hours, and then diluted with 100 μL heptane prior to analysis of intracellular fatty aldehyde and/or fatty alcohol contents by GC-FID or GC-MS.

For extraction and identification of extracellular fatty aldehydes and fatty alcohols, 1 mL of 1:1 (vol:vol) chloroform:methanol is added to 0.5 mL of culture supernatant, and the mixture is vortexed for approximately 30 minutes, and then centrifuged for 2 minutes at 14,000 RPM using a bench top centrifuge. The upper phase is discarded and the approximately 1 mL of the lower phase is transferred to a 2 mL autosampler vial. The extracts are dried under a nitrogen stream, and the residue is derivatized with 100 μL BSTFA at 37-60° C. for 1 hour and then held at room temperature for another 3 to 12 hours. The mixture is diluted with 100 μL heptane prior to analysis of extracellular fatty aldehydes and/or fatty alcohols by GC-FID or GC-MS.

In an exemplary GC-FID or GC-MS procedure, a 1 μL sample is analyzed with the split ratio 1:10, using the following GC parameters: initial oven temperature 80° C. and holding at 80° C. for 3 minutes. The oven temperature is increased to 200° C. at a rate of 50° C./minute, followed by a rate of increase of 10° C./minute to 270° C., and then 20° C./minute to 300° C., followed by a holding at 300° C. for five minutes.

### EXAMPLE 7

This example demonstrates a technique for production of fatty aldehydes and fatty alcohols in Yarrowia lipolytica.

In order to provide for the expression of EntD and CarB in Y. lipolytica, an autonomous replicating plasmid for expression of genes in Y. lipolytica is firstly engineered with antibiotic selection marker cassettes for resistance to hygromycin and phleomycin (HygB® or Ble®, respectively), to generate a plasmid termed “pYLIP.” In pYLIP, expression of each antibiotic selection marker cassette is independently regulated by a strong, constitutive promoter isolated from Y. lipolytica, namely pTEF1 for Ble^{R} expression and pRPS7 for HygB^{R} expression. In pYLIP, heterologous gene expression is under control of the constitutive TEF1 promoter, and the hygB^{R} gene allows for selection in media containing hygromycin. pYLIP also contains an Ars 18 sequence, which is an autonomous replicating sequence isolated from Y. lipolytica genomic DNA. The pYLIP plasmid is then used to assemble Y. lipolytica expression plasmids. Using “restriction free cloning” methodology, an entD gene (e.g., SEQ ID NO: 2) and a gene encoding a CarB polypeptide (e.g., SEQ ID NO: 12) are inserted into pYLIP, thereby generating plasmid “pYLIP1.” pYLIP1 is then transformed by standard procedures into Y. lipolytica 1345, which can be obtained from the German Resource Centre for Biological Material (DSMZ).

To determine the in vivo activity of CarB in recombinant Y. lipolytica host cells, recombinant Y. lipolytica strains expressing EntD and CarB from pYLIP are inoculated into 200 mL YPD media containing 500 μg/mL hygromycin. The cultures are grown at 30° C. to an OD₆₀₀ of approximately 4-7. Cells are then harvested by centrifugation and washed with 20 mL of 50 mM Tris-HCl pH 7.5. Extraction and identification of fatty aldehydes and fatty alcohols are performed as described in Example 6.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing a fatty aldehyde or a fatty alcohol in a host cell, comprising:
(a) expressing a polynucleotide sequence encoding a phosphopanthetheinyl transferase (PPTase) comprising an amino acid sequence having at least 80% identity to the amino acid sequence of SEQ ID NO: 1 in the host cell,
(b) culturing the host cell expressing the PPTase in a culture medium under conditions permissive for the production of a fatty aldehyde or a fatty alcohol, and
(c) isolating the fatty aldehyde or fatty alcohol from the host cell, with the proviso that if the polynucleotide sequence encodes an endogenous PPTase, then the endogenous PPTase is overexpressed.

2. The method of claim 1, further comprising expressing a polynucleotide encoding a polypeptide having carboxylic acid reductase activity.

3. The method of claim 2, wherein the polypeptide having carboxylic acid reductase activity is selected from the group consisting of Mycobacterium smegmatis CarA (SEQ ID NO: 11), Mycobacterium smegmatis CarB (SEQ ID NO: 12), Mycobacterium tuberculosis FadD9 (SEQ ID NO: 13), Nocardia sp. NRRL 5646 CAR (SEQ ID NO: 14), Mycobacterium sp. JLS (SEQ ID NO: 15), Streptomyces griseus (SEQ ID NO: 16), and mutants and fragments of any of the foregoing polypeptides.

4. The method of claim 3, wherein the polypeptide having carboxylic acid reductase activity is Mycobacterium smegmatis CarB (SEQ ID NO: 12) or a mutant or fragment thereof.

5. The method of claim 1, wherein the culture medium does not contain iron.

6. The method of claim 1, wherein the culture medium comprises iron.

7. The method of claim 1, further comprising modifying the expression of a gene encoding a polypeptide involved in iron metabolism.

8. The method of claim 7, wherein the gene encodes an iron uptake regulator protein.

9. The method of claim 8, wherein the gene is fur.

10. The method of claim 1, further comprising modifying the expression of a gene encoding a fatty acid synthase in the host cell.

11. The method of claim 10, wherein modifying the expression of a gene encoding a fatty acid synthase comprises expressing a gene encoding a thioesterase in the host cell.

12. The method of claim 1, further comprising expressing a gene encoding an alcohol dehydrogenase in the host cell

13. The method of claim 1, further comprising modifying the host cell to express an attenuated level of a fatty acid degradation enzyme.

14. The method of claim 1, further comprising culturing the host cell in the presence of at least one biological substrate for the polypeptide.

15. The method of claim 14, wherein the biological substrate is a fatty acid.

16. The method of claim 1, wherein the fatty aldehyde or fatty alcohol is a C₆, C₈, C₁₀, C₁₂, C₁₃, C₁₄, C₁₅, C₁₆, C₁₇, or C₁₈ fatty aldehyde or a C₆, C₉, C₁₀, C₁₂, C₁₃, C₁₄, C₁₅, C₁₆, C₁₇, or C₁₈ fatty alcohol.

17. The method of claim 1, wherein the fatty aldehyde or fatty alcohol is an unsaturated fatty aldehyde or an unsaturated fatty alcohol.

18. The method of claim 17, wherein the unsaturated fatty aldehyde or unsaturated fatty alcohol is C10:1, C12:1, C14:1, C16:1, or C18:1.

19. The method of claim 1, wherein the fatty aldehyde or fatty alcohol is isolated from the extracellular environment of the host cell.

20. The method of claim 1, wherein the host cell is selected from the group consisting of a mammalian cell, plant cell, insect cell, fungus cell, and bacterial cell.

21. The method of claim 1, wherein the polynucleotide sequence encodes an endogenous PPTase, and expression of the polynucleotide sequence is controlled by an exogenous regulatory element.

22. The method of claim 21, wherein the exogenous regulatory element comprises a promoter sequence operably linked to the polynucleotide sequence encoding a PPTase.

23. The method of claim 1, wherein the host cell is E. coli MG1655, the polynucleotide sequence encodes a PPTase consisting of the amino acid sequence of SEQ ID NO: 1, and expression of the polynucleotide sequence is controlled by an exogenous regulatory element.

24. The method of claim 23, wherein the exogenous regulatory element is a promoter sequence operably linked to the polynucleotide sequence encoding a PPTase.

25. A fatty aldehyde produced by the method of claim 1.

26. A fatty alcohol produced by the method of claim 1.

27. A surfactant comprising the fatty alcohol of claim 26.

28.-43. (canceled)
